(19) United States
(12) Patent Application Publication
(10) Pub. No.: US 2008/0176225 A1
(43) Pub. Date: Jul. 24, 2008

(54) MEMBRANE BOUND REPORTER GENE SYSTEM

(76) Inventors: Steve ROFFLER, Taipei (TW); Tian-Lu Cheng, Kaohsiung City (TW); Yu-Cheng Su, Taipei City (TW)

Correspondence Address: PATTERSON & SHERIDAN, L.L.P., 3040 POST OAK BOULEVARD, SUITE 1500, HOUSTON, TX, 77056, US

(21) Appl. No.: 11/624,625

(22) Filed: Jan. 18, 2007

(51) Int. Cl. C12Q 1/68; C07H 21/04; C12N 15/55; C12N 15/85

(52) U.S. Cl. 435/6; 435/320.1; 435/455; 536/23.1; 536/23.2; 536/23.72

(57) ABSTRACT

A recombinant DNA construct is provided and includes a first DNA fragment encoding a β-glucuronidase and a second DNA fragment encoding a membrane anchoring domain. The β-glucuronidase may be a human β-glucuronidase or a mouse β-glucuronidase. In one embodiment, an expression vector for delivering a gene of interest or portion thereof into a host cell includes a DNA sequence for the gene of interest, a first DNA fragment encoding a β-glucuronidase, and a second DNA fragment encoding a membrane anchoring domain. In another embodiment, a method of introducing a gene of interest or portion thereof into a host cell is provided, including introducing into the host cell a recombinant DNA construct.

### BACKGROUND OF THE INVENTION

Technological advances in reporter gene systems have enabled the introduction of various genes in vitro and in vivo into different kinds of cells, and even into whole organisms. Reporter genes, such as β-galactosidase (β-gal), chloramphenicol acetyltransferase, herpes simplex type 1 virus thymidine kinase, luciferase, green fluorescent protein, cytosine deaminase, and other proteins have been used to study gene expression and regulation in biological systems. In addition to facilitate exogenous gene expression, reporter genes have found many applications in basic research and biotechnology. For example, introducing genes into organisms for therapeutic purposes, gene therapy, has been described as the fourth revolution in medicine. Currently, many research centers and biotechnology companies have focused on developing gene vectors to deliver therapeutic genes in vivo into targeted cells and tissues.

In general, reporter gene systems allow for the measurement of gene expression through measurements of final products of enzymatic reactions and/or the expression of bioluminescent or fluorescent proteins. However, expression of many reporter genes and thus their exogenous gene products in animals can induce immune responses that result in tissue damage and limit persistent gene expression and imaging. To be clinically useful, a reporter gene should display low immunogenicity to allow repeated administration and prolonged expression; however, most in vivo reporter genes are derived from non-endogenous sources and induce both cellular and humoral immune responses. Endogenous reporter genes such as the dopamine D2 receptor and the transferrin receptor are less immunogenic but suffer from poor specificity due to their widespread expression.

In addition, measuring expression of many available reporter gene systems requires destroying host cells, performing biopsies, or killing the animal to recover tissues. Further, the reporter gene should be specific to allow unambiguous identification of the location and extent of gene expression. Sensitive and specific reporter genes are thus needed for the continued development of transgenic animals and the practice of gene therapy in human and others organisms.

Therefore, development of a non-invasive reporter gene that can trace the gene expression in vivo at different resolutions, sensitivities, costs, and quantities would certainly improve vector usage to achieve better success in gene therapy suitable for many disease interventions and cancer therapy.

### SUMMARY OF THE INVENTION

Embodiments of the invention generally provides a recombinant DNA construct having a first DNA fragment encoding a β-glucuronidase and a second DNA fragment encoding a membrane anchoring domain. The membrane anchoring domain may be a simple anchoring domain, an anchor, such as a glycosylphosphatidylinositol (GPI) anchor, or a transmembrane domain of an integral membrane protein. A GPI anchor may be derived from, for example, decay accelerating factor, CDw52, CD55, CD59 and thy-1, and combinations thereof. The integral membrane protein may be, for example, type I integral membrane proteins, type II integral membrane proteins, type III integral membrane proteins, membrane bound receptor proteins, a murine B7-1 antigen (e-B7), platelet-derived growth factor receptor (PDGFR), intracellular adhesion molecule 1 (ICAM-1), asialoglycoprotein receptor (ASGPR), aminopeptidase N (CD13), mast-cell function-associated antigen, influenza virus neuraminidase, dipeptidyl aminopeptidase IV (CD26), and combinations thereof. The β-glucuronidase may be a human β-glucuronidase, a mouse β-glucuronidase, or an E. coli β-glucuronidase. β-glucuronidase from other species may be suitable. The anchoring domain may be, for example, a GPI (glycosylphosphatidylinisotol) anchor and other anchors.

In one embodiment, a method of introducing a gene of interest or portion thereof into a host cell is provided, including introducing into the host cell a recombinant DNA construct having a DNA sequence for the gene of interest or portion thereof, a first DNA fragment encoding a β-glucuronidase, and a second DNA fragment encoding a transmembrane domain of an integral membrane protein.

In another embodiment, an expression vector for delivering a gene of interest or portion thereof into a host cell includes a DNA sequence for the gene of interest, a first DNA fragment encoding a β-glucuronidase, and a second DNA fragment encoding a transmembrane domain of an integral membrane protein. The DNA sequence for the gene of interest may include a DNA fragment encoding a product of the gene of interest and/or a regulatory DNA region for the expression of the gene of interest, such as promoter DNA regions, tissue-specific regulatory DNA regions, up-expression regulatory DNA regions, and/or down-expression regulatory DNA region for the expression of an exogenous gene of interest.

In still another embodiment, a method of imaging the expression of a gene of interest in a host cell includes introducing into the host cell a recombinant DNA construct, the recombinant DNA construct comprising a DNA sequence for the gene of interest or portions thereof, a first DNA fragment encoding a β-glucuronidase, and a second DNA fragment encoding a transmembrane domain of an integral membrane protein. The method further includes providing a non-fluorescent substrate capable of be converted into a fluorescent report product by the β-glucuronidase and monitoring the levels of the fluorescent reporter product in the host cell.

### BRIEF DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1A illustrates an exemplary membrane bound reporter transgene having a β-glucuronidase (βG) gene linked to a membrane anchoring domain (MA), such as an anchor or a transmembrane (TM) domain, according to one embodiment of the invention.

FIG. 1B illustrates another exemplary membrane bound reporter transgene having a β-glucuronidase gene and a membrane anchoring domain (MA), such as a transmembrane (TM) domain of a type II integral membrane protein linked to N-terminal of the β-glucuronidase (βG) gene, according to another embodiment of the invention.

FIG. 1C illustrates an exemplary membrane bound reporter transgene having a β-glucuronidase gene linked to a spacer and a membrane anchoring domain (MA), such as a TM domain of an integral membrane protein, according to another embodiment of the invention.

FIG. 1D illustrates an exemplary membrane bound reporter transgene having a β-glucuronidase gene linked to a membrane anchoring domain (MA) and a cytosolic domain according to another embodiment of the invention.

FIG. 1E illustrates an exemplary membrane bound reporter transgene having a β-glucuronidase gene linked to a membrane anchoring domain (MA) and a leader sequence domain according to another embodiment of the invention.

FIG. 1F illustrates an exemplary membrane bound reporter transgene having a β-glucuronidase gene linked to a membrane anchoring domain (MA) and an epitope according to another embodiment of the invention.

FIG. 1G illustrates an exemplary membrane bound reporter transgene having an epitope and a β-glucuronidase gene linked to a membrane anchoring domain (MA) according to another embodiment of the invention.

FIG. 1H illustrates an exemplary membrane bound reporter transgene having a β-glucuronidase gene linked to a membrane anchoring domain (MA) and a linker amino acid sequence and/or a spacer domain according to another embodiment of the invention.

FIG. 1I illustrates an exemplary membrane bound reporter transgene having a leader sequence, a β-glucuronidase gene linked to a spacer domain, a membrane anchoring domain (MA), and a cytosolic domain according to another embodiment of the invention.

FIG. 2 illustrates an exemplary membrane-bound β-glucuronidase reporter system anchored on the outer surface of the cellular plasma membrane to hydrolyze enzymatically the glucuronide group of a non-fluorescent substrate (FDGlcU) into a highly fluorescent compound (fluorescein) according to one embodiment of the invention.

FIG. 3 illustrates an exemplary membrane-bound β-glucuronidase (βG) reporter system having cDNA sequences encoding for an immunoglobulin kappa chain leader sequence (LS) followed by an HA epitope (HA), the mature β-glucuronidase (βG) gene, a myc epitope (myc), the immunoglobulin-like C2-type extracellular region (B7 spacer domain) of B7-1 in addition to the transmembrane (TM) domain and the cytosolic domain of a murine B7-1 gene, where the gene expression is under the control of a CMV promoter, according to one embodiment of the invention.

FIG. 4 shows cells surface display of an exemplary functional mouse membrane bound β-glucuronidase reporter system expressed in live mouse CT26 cells with and without murine β-glucuronidase-eB7 transgene according to one embodiment of the invention.

FIG. 5 shows the results of in vivo imaging of an exemplary functional mouse membrane bound β-glucuronidase reporter system using FDGlcU as its substrate, which is intravenously injected into mice bearing CT26 tumors (left) and CT26/mβG-eB7 (right) tumors with whole-body images acquired at the indicated times of 3 mins, 30 mins, 60 mins according to one embodiment of the invention.

FIG. 6 shows the pharmacokinetics results of FDGlcU activation using the exemplary system of FIG. 5 according to one embodiment of the invention. The CT26 (open circle) and CT26/mβG-eB7 (black dot) tumors (n=4) were determined by measuring fluorescence intensities in 3 minute scans performed over 90 minutes according to one embodiment of the invention.

FIG. 7 shows thin cell sections of CT26 (upper panels) and CT26/mβG-eB7 (lower panels) tumors stained with X-GlcA and nuclear fast red (NFR) according to one embodiment of the invention.

FIG. 8 shows the in vivo biodistribution of the reaction product (fluorescein) in mice according to one embodiment of the invention. In vivo biodistribution of the fluorescein was measured 30 minutes after i.v. injection of the substrate, FDGlcU, (left panel) or fluorescein (right panel).

FIG. 9 is a graph showing the comparison of the results of the example of FIG. 8 in different organ regions of interest according to one embodiment of the invention.

FIG. 10 shows the results of in vitro infection of exemplary HCC36 tumor cells (open curve) and Ad5/mβG-eB7 infected HCC36 cells (solid curve) according to one embodiment of the invention.

FIG. 11 shows the example in which a nude mouse bearing HCC36 tumors was injected with adenovirus (Ad5/mβG-eB7) into the HCC36 tumor on the right side of the mouse. The mouse was i.v. injected with FDGlcU and imaged according to one embodiment of the invention.

FIG. 12 shows the results of cell sections of HCC36 tumors (upper panel) and Ad5/mβG-eB7 injected HCC36 tumors (lower panel) according to one embodiment of the invention.

FIG. 13 shows low immunogenicity of an exemplary functional mouse membrane bound β-glucuronidase reporter system as compared to high immunogenicity of a LacZ membrane bound reporter system according to one embodiment of the invention.

FIG. 14 shows the results of testing serum samples to demonstrate low immunogenicity of an exemplary functional mouse membrane bound β-glucuronidase reporter system as compared to high immunogenicity of a LacZ membrane bound reporter system according to one embodiment of the invention.

FIG. 15 shows the analysis and imaging of an exemplary functional human membrane bound β-glucuronidase reporter system according to one embodiment of the invention.

FIG. 16 shows the results of the analysis and imaging of an exemplary functional human membrane bound β-glucuronidase reporter system i.v. injected into nude mice capable of infecting mouse cells and developing tumors according to one embodiment of the invention.

FIG. 17 shows another exemplary membrane bound β-glucuronidase reporter system according to one embodiment of the invention.

FIG. 18 illustrates various suitable spacer domains and transmembrane domains according to embodiments of the invention.

FIG. 19 illustrates immunoblots of exemplary membrane-bound β-glucuronidase reporter systems expressed in 3T3 fibroblast cells according to one embodiment of the invention.

FIG. 20 illustrates immunoblots of exemplary membrane-bound β-glucuronidase reporter systems expressed in 3T3 fibroblast cells according to another embodiment of the invention.

FIG. 21 shows the results of immunofluorescence as analyzed on a flow cytometer by expressing various exemplary functional membrane bound β-glucuronidase reporter systems in 3T3 fibroblast cells according to embodiments of the invention.

FIG. 22 shows the immunofluorescence as analyzed on a flow cytometer or β-glucuronidase enzyme activity of expressed membrane bound human β-glucuronidase recombinant constructs in 3T3 fibroblast cells according to embodiments of the invention.

FIG. 23 shows the immunofluorescence as analyzed on a flow cytometer or β-glucuronidase enzyme activity of expressed membrane bound mouse β-glucuronidase recombinant constructs in 3T3 fibroblast cells according to embodiments of the invention.

FIG. 24 shows the immunofluorescence as analyzed on a flow cytometer or β-glucuronidase enzyme activity of expressed membrane bound E. coli β-glucuronidase recombinant constructs in 3T3 fibroblast cells according to embodiments of the invention.

FIG. 25 shows the immunoblot results of characterization of exemplary membrane-bound β-glucuronidase expression in stable EJ bladder carcinoma cells according to one embodiment of the invention.

FIG. 26 shows the immunofluorescence as analyzed on a flow cytometer of various exemplary functional membrane-bound β-glucuronidase reporter systems in live EJ cells according to embodiments of the invention.

FIG. 27 shows the immunofluorescence as analyzed under a fluorescence microscope equipped with a CCD detector (upper panels) or under phase-contrast (lower panels) of various exemplary functional membrane bound β-glucuronidase reporter systems in live EJ cells according to embodiments of the invention.

FIG. 28 shows the results of β-glucuronidase enzyme activity of various exemplary membrane bound β-glucuronidase recombinant constructs in EJ cells according to embodiments of the invention.

FIG. 29 shows an SDS-PAGE gel electrophoresis of the purified human, mouse, and E. coli β-glucuronidase recombinant proteins according to an embodiment of the invention.

FIG. 30 shows relative enzymatic activities of the purified human, mouse, and E. coli β-glucuronidase recombinant proteins at the indicated pH values (n=3) according to an embodiment of the invention.

FIG. 31 shows the specific activities of the recombinant human, mouse, and E. coli β-glucuronidase proteins, at the indicated pH values according to an embodiment of the invention.

FIG. 32 illustrates linking of an exemplary transmembrane domain from a type II integral membrane protein, ASGPR, to E. coli β-glucuronidase and expressing the membrane-bound β-glucuronidase in 3T3 cells according to one embodiment of the invention.

FIG. 33 shows the results of the glucuronidase activity for various exemplary recombinants constructs as shown in FIG. 32 according to one embodiment of the invention.

FIG. 34 shows the immunofluorescence as analyzed on a flow cytometer of an exemplary functional membrane anchored β-glucuronidase reporter system with a GPI anchor in live BHK cells according to embodiments of the invention.

FIG. 35 shows the immunofluorescence as analyzed on a flow cytometer of live BHK cells without a β-glucuronidase reporter system for comparison according to one embodiment of the invention.

FIG. 36 shows in vivo imaging by targeted activation of a glucuronide TRAP compatible substrate/probe, difluoromethylphenol-¹²⁴I glucuronide (¹²⁴I-trap-glu), which can be enzymatically converted to an active trap-¹²⁴I by membrane bound β-glucuronidase to assess the location and persistence of gene expression in vivo, according to one embodiment of the invention.

FIG. 37 illustrates the chemical structure of the ¹²⁴I-difluoromethylphenol glucuronide probe (¹²⁴I-trap-glu) according to one embodiment of the invention.

FIG. 38 shows the results demonstrating the specificity of mβG-eB7, which specifically converts the ¹²⁴I-difluoromethylphenol glucuronide probe (¹²⁴I-trap-glu) to ¹²⁴I-trap product in CT26/mβG-eB7 (▪) but not in CT26 cells (□) according to one embodiment of the invention.

FIG. 39 shows the results of in vivo micro-PET (Positron Emission Tomography) imaging of membrane bound β-glucuronidase gene expression by ¹²⁴I-trap-glu according to one embodiment of the invention.

FIG. 40 illustrates the chemical structure of the ¹²⁴I-phenolphthalin glucuronide probe (¹²⁴I-ph-trap-glu) according to one embodiment of the invention.

FIG. 41 shows the results demonstrating the specificity of mβG-eB7, which specifically converts the ¹²⁴I-phenolphthalein glucuronide probe (¹²⁴I-ph-trap-glu) to ¹²⁴I-trap product in CT26/mβG-eB7 (▪) but not in CT26 cells (□) according to one embodiment of the invention.

FIG. 42 shows the results of in vivo micro-PET imaging of membrane bound β-glucuronidase gene expression by ¹²⁴I-ph-trap-glu according to one embodiment of the invention.

FIG. 43 shows the results demonstrating specific trapping of FITC-trap-glu by mβG-eB7 as detected by anti-FITC antibody but not anti-BSA antibody according to one embodiment of the invention.

FIG. 44 shows the results of the measured β-glucuronidase activities at different concentrations of FITC-trap-glu according to one embodiment of the invention.

FIG. 45 shows the results demonstrating the specificity of FITC-trap-glu activation in β-glucuronidase-expressing cells in vitro as detected by anti-FITC antibody and observed under phase contrast and fluorescent field confocal microscope according to one embodiment of the invention.

FIG. 46 shows the results of demonstrating the specificity of FITC-trap-glu activation in β-glucuronidase-expressing cells in vivo as detected by iv injection the substrate FITC-trap-glu and observed under phase contrast and fluorescent field confocal microscope according to one embodiment of the invention.

### DETAILED DESCRIPTION

Embodiments of the present invention generally relate to DNA constructs useful for monitoring gene expression. In one embodiment, a novel reporter gene system, which anchors an enzymatic gene product to the surface of cell membranes and allows extracellular hydrolysis of a non-chromogenic probe/substrate to a chromogenic product by the enzymatic gene product is provided. For example, the novel reporter gene system may include a membrane bound reporter enzyme capable of converting a non-fluorescent probe/substrate to a fluorescent product by linking to a membrane anchoring domain.

The membrane anchoring domain may be an anchoring domain, such as a GPI anchor, or a transmembrane (TM) domain of an integral membrane protein. One example of the novel reporter gene system may include a gene product of a β-glucuronidase gene linked to a membrane anchoring domain, including an anchor domain or a transmembrane domain of an integral membrane protein. One example of an anchor domain may be a GPI anchor (glycosylphosphatidylinisotol anchor).

The gene product of the β-glucuronidase gene is demonstrated herein to be assembled into a functionally active tetramer β-glucuronidase enzyme when expressed and bound to the cell membrane by anchoring/linking through the membrane anchoring domain and thus to hydrolyze and convert non-chromogenic substrates into chromogenic, fluorescent, or radioactive products. Accordingly, the DNA constructs of the novel reporter gene system described herein can be used as a non-invasive reporter gene construct that can trace in vivo gene expression of any known or unknown gene of interest at very high resolution, high sensitivity, low cost, and high levels to achieve better success in gene therapy, disease interventions, and cancer therapy, etc., in human and other organisms.

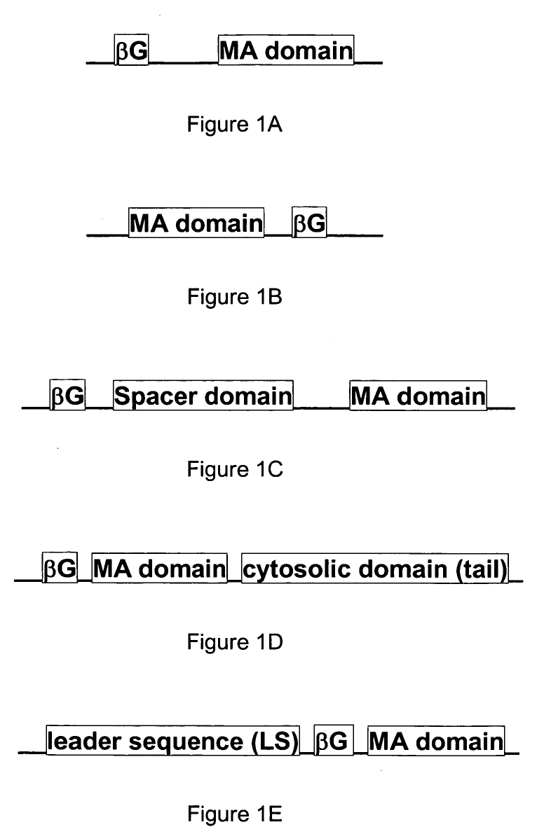

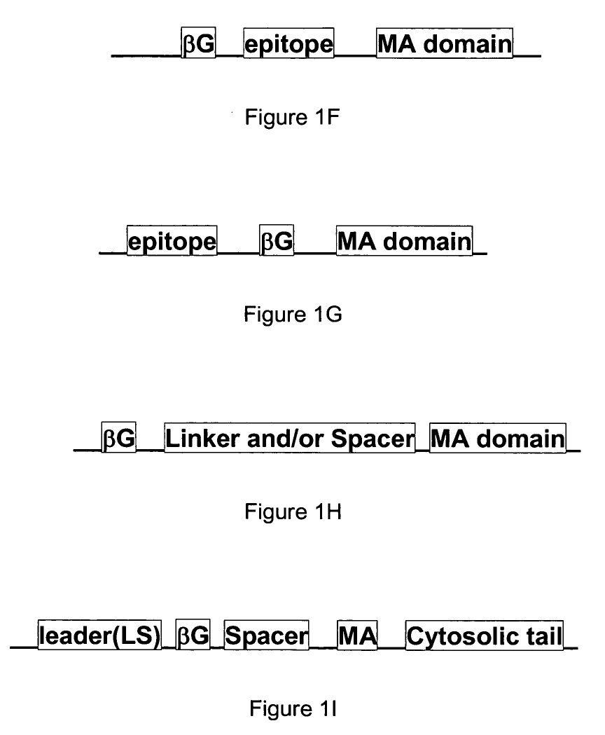

FIG. 1A illustrates an exemplary membrane bound reporter system having a β-glucuronidase gene linked to a membrane anchoring domain (shown as MA in FIGS. 1A-1I) for efficient transport of the enzymatic β-glucuronidase gene product to the cell surface. Suitable β-glucuronidase genes include, but are not limited to, a human β-glucuronidase gene, a mouse β-glucuronidase gene, an E. coli β-glucuronidase (GUS) gene, among others Other β-glucuronidase genes, such as those from bovine, dog, cat may also be used by cloning the β-glucuronidase genes and testing their activity as a membrane bound enzyme. Other enzymatic gene products capable of converting its substrate to a product that can be detected, assayed, or imaged can also be used. For example, DNA fragments for other enzymes capable of converting a non-chromogenic probe/substrate to a chromogenic product, such as converting a non-fluorescent probe/substrate to a fluorescent product, can also be used.

Suitable DNA fragments for eukaryotic enzymes, such as eukaryotic β-glucuronidases, may have a leader sequence to allow transport of the protein into the endoplasmic reticulum for export to the cell surface. However, when DNA fragments of prokaryotic enzymes are used; a leader sequence from another protein can be additionally inserted into the membrane bound reporter system. For example, an E. coli β-glucuronidase gene may be fused to a mammalian leader sequence at its 5′ end (to the N-terminus of its protein), since a leader sequence is mandatory to allow transport of the protein into the endoplasmic reticulum for export to the cell surface. In some cases, such as TM domains of a Type II membrane protein, the domains of the Type II membrane protein may include a leader sequence such that a leader sequence present in the β-glucuronidase may be optional.

The transmembrane domain can be derived from any integral membrane protein. For example, the integral membrane proteins may be, but not limited to, type I integral membrane proteins (with the N-terminus oriented extracellularly or outside a cell), type II integral membrane proteins (with the N-terminus oriented in the cytosol), and type III integral membrane proteins (with multiple membrane spanning regions), among others. Exemplary type I integral membrane proteins that can be successfully linked in-frame to β-glucuronidase (βG) cDNA include platelet-derived growth factor receptor (PDGFR), B7-1 antigen (B7), and intracellular adhesion molecule 1 (ICAM-1), etc.

Alternatively, β-glucuronidase can be anchored via a GPI (glycosylphosphatidylinisotol) anchor and other anchors. Examples of GPI anchors suitable for attaching β-glucuronidase on cells include decay accelerating factor, CDw52, CD55, CD59 and thy-1.

FIG. 1B illustrates another exemplary membrane bound reporter transgene having a β-glucuronidase cDNA and a membrane anchoring domain (MA), such as a transmembrane (TM) domain of a type II integral membrane protein, linked to N-terminal of the β-glucuronidase cDNA. Exemplary type II integral membrane proteins that can be successfully linked in-frame to β-glucuronidase cDNA includes asialoglycoprotein receptor (ASGPR), aminopeptidase N (CD13), mast-cell function-associated antigen, influenza virus neuraminidase, dipeptidyl aminopeptidase IV (CD26) or any type II transmembrane domains.

It is found that a human β-glucuronidase gene, a mouse β-glucuronidase gene, and an E. coli β-glucuronidase gene are herein successfully linked to many membrane anchoring domains, such as a TM domain of a mouse B7-1 antigen (e-B7), human platelet-derived growth factor receptor (PDGFR), human intracellular adhesion molecule 1 (ICAM-1), and human asialoglycoprotein receptor (ASGPR), etc., and anchored to the cell surface. TM domains from various membrane proteins and GPI anchor are tested and are found to compatible with the activity of the linked β-glucuronidase. In general, a membrane anchoring domain, such as an TM domain or an membrane anchor, which allows the proper folding and does not interfere with the enzymatic activities of the enzymatic gene products of β-glucuronidase can be used.

The DNA fragments for β-glucuronidases and the transmembrane domains can be linked together by fusing the DNA fragments encoding the β-glucuronidase and the transmembrane domain in-frame transcriptionally to allow production of a single polypeptide after translation of the polypeptide containing these DNA fragments into mRNA.

The novel reporter gene system may further include a spacer domain between the enzymatic gene product and the transmembrane domain. FIG. 1C illustrates an exemplary membrane bound reporter transgene having a β-glucuronidase (βG) gene linked to a spacer domain and a membrane anchoring domain. The spacer domain is designed to be useful for optimum spacing and proper three-dimensional membrane-bound protein folding. The spacer domain can also include an epitope designed to be useful for monitoring and detection of the expression of the novel reporter gene system.

The spacer domain can be a few amino acids long, a peptide, a long polypeptide chain, or a full length protein as long as the spacer domain does not interfere with the enzymatic activity of the enzymatic gene product of β-glucuronidase genes or the proper folding of the transmembrane domain. In addition, suitable spacer domains allow more flexible assembly of the enzymatic gene product of β-glucuronidase genes into a β-glucuronidase tetramer. For example, a myc epitope or a HA epitope can be inserted between an β-glucuronidase gene and a transmembrane (TM) domain of an integral membrane protein. Detection or monitoring of the expression of the novel reporter gene system can thus be performed using, for example, an antibody for a chosen suitable epitope, such as an antibody for myc epitope. Exemplary epitopes include myc epitope, HA epitope, flag epitope, a flexible polypeptide, or any epitope that can be bound or detected by a suitable antibody.

The spacer domain may also be, but is not limited to, one or more extracellular domains of an integral membrane protein, which can be the same, or a different membrane protein from the membrane protein for the TM domain. For example, the Ig-like C2-type and Ig-hinge-like domains (e) of murine CD80 (B7-1 protein), the hinge-CH2-CH3 domains of a human immunoglobulin IgG1, the CH2-CH3 domains (lacking the hinge domain) of a human IgG1, the N-terminal Ig-like V-type domain of human biliary glycoprotein-1 (BGP-1), a BGP1 extracellular protein domain, a myc epitope, a HA epitope, a flag epitope, a flexible polypeptide, an extracellular domain of a membrane protein, an extracellular domain of the constant domain of a mouse B7 protein, and the extracellular portion of human CD44E, etc., can be successfully fused and linked herein to a human β-glucuronidase gene, a mouse β-glucuronidase gene, or an E. coli β-glucuronidase gene to allow more flexible assembly of the βG tetramer.

Further, the spacer domain may contain one or more glycosylation sites and/or polysaccharide chains to reduce the shedding of the chimeric membrane bound reporter transgene protein from the cell surface. The glycosylation sites useful for linking polysaccharides or oligosaccharides may be O-linked or N-linked glycosylation sites. Oligosaccharides or polysaccharides present in the juxtamembrane spacer domain may help reduce shedding of the membrane bound β-glucuronidase. For example, the spacer domain can be present and/or inserted between β-glucuronidase and the transmembrane domain, and can be derived from segments of the ectodomains of any membrane proteins, but optionally should contain polysaccharide chains. The carbohydrates are provided to protect proteolytic cleavage of the enzyme from the cell surface.

The novel reporter gene system may further include one or more cytosolic domains of an integral membrane protein, which can be from the same, or a different membrane protein from the membrane protein for the TM domain. FIG. 1D illustrates an exemplary membrane bound reporter transgene having a β-glucuronidase gene linked to a TM domain of an integral membrane protein and a cytosolic domain. In general, any cytosolic domain of an integral membrane protein can be used as long as it may help the proper folding of the integral membrane protein and/or provide proper transport of the recombinant protein product of the novel reporter gene system to the cell membrane. In addition, a cytosolic domain of an integral membrane protein, which does not interfere with the enzymatic activity of the enzymatic gene product of β-glucuronidase genes, can be used. The cytosolic tail/domain helps to transport the resulting recombinant protein to the cell surface faster and can therefore allow higher levels of expression of the membrane bound reporter enzyme on the cell surface.

Leader sequence (LS) derived from any suitable secreted or membrane proteins can also be inserted into the reporter gene system to direct the expression and transport of the recombinant membrane bound β-glucuronidase. FIG. 1E illustrates another example of a membrane bound reporter system having a leader sequence domain in front of a β-glucuronidase (βG) cDNA linked to a TM domain of an integral membrane protein.

FIG. 1F illustrates yet another membrane bound reporter system having a β-glucuronidase cDNA linked to a membrane anchoring domain, such as a TM domain of an integral membrane protein, and an epitope according to another embodiment of the invention. The epitope can be linked after the β-glucuronidase cDNA and/or in front of the β-glucuronidase cDNA. For example, FIG. 1G illustrates a membrane bound reporter system having an epitope tagged in front of the β-glucuronidase cDNA and linked to a membrane anchoring domain. In some cases where the epitope is linked in front of the β-glucuronidase cDNA, a DNA fragment of a suitable leader sequence may also be inserted to the 5′ end of the epitope.

Artificial or synthetic linker sequence can also be inserted into the reporter gene system to optimize the expression levels of the recombinant membrane bound β-glucuronidase and/or stabilize the recombinant membrane bound β-glucuronidase protein For example, a stretch (a few amino acid long) of small amino acids, such as glycine, serine, and other amino acid, etc., can be used as linker sequence. FIG. 1H illustrates an exemplary membrane bound reporter system having a β-glucuronidase (βG) cDNA linked to a TM domain of an integral membrane protein and a linker amino acid sequence and/or a spacer domain fused in between.

FIG. 1I illustrates another exemplary membrane bound reporter system having a leader sequence, a β-glucuronidase gene linked to a spacer domain, a membrane anchoring domain, and a cytosolic domain to optimize the expression levels of the membrane bound reporter system and anchor the membrane bound reporter system to the surfaces of the cell membranes. The recombinant DNA constructs as exemplarily shown in FIGS. 1A-1I may be used individually or in combination. For example, the leader sequence as shown in FIG. 1E can be combined with the recombinant DNA construct of FIG. 1C and inserted in front of the β-glucuronidase cDNA to generate a resulting recombinant DNA construct and thus synthesize a recombinant protein for the novel reporter gene system which includes a leader sequence, a β-glucuronidase, a spacer domain, and a TM domain. As another example, the leader sequence as shown in FIG. 1E can be combined with the recombinant DNA construct of FIG. 1D and inserted in front of the β-glucuronidase cDNA to generate a resulting recombinant DNA construct and thus synthesize a recombinant protein for the novel reporter gene system which includes a leader sequence, a β-glucuronidase, a spacer domain, and a TM domain.

The spacer domain and the cytosolic domain, which may or may not directly assist cell surface expression, may be optionally linked to the recombinant membrane bound reporter construct to assist the expression of the recombinant β-glucuronidase-transmembrane domain construct and/or enhance the stability of the recombinant protein having β-glucuronidase linked to a transmembrane domain

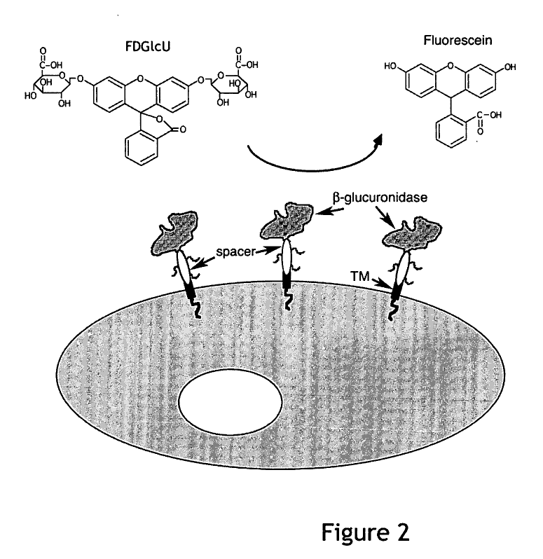

FIG. 2 illustrates an exemplary membrane-bound β-glucuronidase reporter system anchored on the outer surface of the cellular plasma membrane to hydrolyze enzymatically the glucuronide group of a non-fluorescent substrate (FDGlcU) into a highly fluorescent compound (fluorescein) according to one embodiment of the invention. By anchoring the β-glucuronidase enzyme on the surface of cell membranes, non-fluorescent glucuronide substrates/probes are able to undergo extracellular hydrolysis into fluorescent reporter products by the membrane bound β-glucuronidase enzyme. Accordingly, there is no need to transport the non-fluorescent glucuronide substrates/probes across the membrane barrier of a cell to be inside the cell, which, in some cases, may be toxic to the cell. In addition, there is no need to break down the cell or lyse the cell for the β-glucuronidase to hydrolyze the non-fluorescent glucuronide substrates/probes and thus allow in vivo monitoring and imaging of the fluorescent compound/product to be feasible.

The novel membrane bound reporter gene system is designed to retain substrate specificity of the reporter gene product when displayed on the cell surface and is shown herein to display low immunogenicity. The endogenous β-glucuronidase enzyme is normally expressed in lysosomes and the substrates of the lysosomal β-glucuronidase enzyme can not normally enter through the cell membrane without the help of a membrane permease or transferase. It is shown herein that the activity of the membrane-bound β-glucuronidase reporter system is not interfered by any endogenous or lysosomal β-glucuronidase, if any is present.

It is contemplated that substrate specificity of the membrane-bound β-glucuronidase could be retained since endogenous β-glucuronidase is located in lysosomes and only very low levels of β-glucuronidase are found in human serum. Most of its glucuronide substrates are charged at physiological pH values which hinder their diffusion across the lipid bilayer of cell membranes, effectively sequestering any glucuronide substrates/probes from contact with endogenous lysosomal β-glucuronidase. Conjugation of glucuronide moieties to xenobiotics by an UDP-glucuronosyl transferase is also a major detoxification pathway in rodents and humans, suggesting that a glucuronide probe should be resistant to premature activation by endogenous lysosomal β-glucuronidase under physiological conditions.

Accordingly, non-immunogenic, non-invasive, and substrate-specific reporter gene systems are developed. A membrane-anchored form of β-glucuronidase can be used as a reporter gene system to facilitate persistent gene expression into various host cells and/or access the location of the expression of exogenous foreign genes. The β-glucuronidase on the surface of cells is functionally active in converting its substrates, such as a non-fluorescent glucuronide probe (fluorescein di-β-D-glucuronide, FDGlcU) to a highly fluorescent reporter product on the cell surface in vivo.

In one embodiment, a recombinant DNA construct is provided and includes a first DNA fragment encoding a β-glucuronidase and a second DNA fragment encoding a transmembrane domain of an integral membrane protein. The recombinant DNA construct may also include a DNA fragment of a spacer domain and/or one or more glycosylation sites. The recombinant DNA construct may further include a DNA fragment of a cytosolic domain of a membrane protein.

In another embodiment, the recombinant DNA construct may further include a DNA fragment encoding a product of an exogenous gene of interest. In still another embodiment, the recombinant DNA construct may also include a regulatory DNA region, such as promoter DNA regions, tissue-specific regulatory DNA regions, up-expression regulatory DNA regions, and/or down-expression regulatory DNA region for the expression of an exogenous gene of interest.

According to one or more embodiments of the invention, a method of introducing a gene of interest or portion thereof into a host cell is provided to deliver and introduce a recombinant DNA construct into the host cell. The recombinant DNA construct suitable for gene therapy or gene delivery may include a DNA sequence for the gene of interest or portion thereof, a first DNA fragment encoding a β-glucuronidase, and a second DNA fragment encoding a transmembrane domain of an integral membrane protein. The DNA sequence for the gene of interest may include a DNA fragment encoding a product of the gene of interest and/or a regulatory DNA region for the expression of the gene of interest.

The host cell can be any suitable cells, tissues, and/or cell lines, such as tumors, tumor cell lines, fibroblast cell lines, among others. The gene of interest or portion thereof may be introduced into the host cell by, but not limiting to, direct injection into tissues or tumors, delivery as part of a liposomal formulation to specific tissues or organs after addition by various routes, such as direct tissue injection, subcutaneous (s.c.), intravenous (i.v.), intraperitoneal (i.p.), intrahepatic (i,h,) injections etc, infection using viral delivery vectors administrated by i.p., s.c, i.v. etc., routes, and/or hydrodynamic administration, among other routes.

Another embodiment of the invention provides an expression vector for delivering a gene of interest or portion thereof into a host cell. The expression vector may include a DNA sequence for the gene of interest, a first DNA fragment encoding a β-glucuronidase, and a second DNA fragment encoding a transmembrane domain of an integral membrane protein. The expression vector may also include a DNA fragment of a spacer domain and/or one or more glycosylation sites. The expression vector may further include a DNA fragment of a cytosolic domain of a membrane protein. The β-glucuronidase is capable of converting a non-fluorescent substrate to a fluorescent report product.

### Applications for Membrane Bound Reporter Gene Systems

The novel reporter gene system may be useful by constructing as a vector-alone control or as a vector for inserting one or more known or unknown genes and/or regulatory DNA fragments/regions from one or more known or unknown genes. For example, the novel reporter gene system having the β-glucuronidase linked to a TM domain can be constructed into an expression vector for delivery known DNA sequences for monitoring the expression of the known DNA sequences, such as by monitoring and/or imaging the levels of the fluorescent product of the hydrolysis of the substrate of the β-glucuronidase by the membrane bound β-glucuronidase reporter system. The novel reporter gene system can also be used for delivery unknown DNA sequences for screening gain of function or loss of function for the expression of the unknown DNA sequences.

As an example, the novel reporter gene system can be used to estimate the efficiency, safety and specificity of different gene delivery systems in vivo and in vitro. The novel reporter gene system can be used to be compatible with gene delivery systems approved by FDA and to accelerate gene delivery systems to be approved by FDA.

The novel reporter gene system can be used for imaging tissue specific promoter gene expression in transgenic animals. The transgene expressing the β-glucuronidase reporter can be placed under the control of a promoter of interest to allow expression of β-glucuronidase to be controlled by the promoter. Transgenic techniques can be employed to develop animals that incorporate the promoter/reporter gene transgene in their cells. A glucuronide imaging agent can then be administered at defined times and gene expression in various tissues and organs can be non-invasively observed in live animals.

The novel reporter gene system can also be used for both imaging and therapy of cancer. Glucuronide prodrugs are attractive for cancer therapy due to their low toxicity, bystander effect in the interstitial tumor space and the large range of possible glucuronide drug targets. We expressed human, murine and E. coli β-glucuronidase on tumor cells and examined their in vitro and in vivo efficacy for the activation of glucuronide prodrugs of 9-aminocamptothecin and p-hydroxy aniline mustard. Fusion of β-glucuronidase to the Ig-like C₂-type and Ig-hinge-like domains of the B7-1 antigen followed by the B7-1 transmembrane domain and cytoplasmic tail anchored high levels of active murine and human β-glucuronidase on cells. Potent in vivo antitumor activity was achieved by prodrug treatment of tumors that expressed murine β-glucuronidase. The p-hydroxy aniline prodrug was more effective in vivo than the 9-aminocamptothecin prodrug. Surface expression of murine β-glucuronidase for activation of a glucuronide prodrug of p-hydroxy aniline mustard may be useful for more selective therapy of cancer. Therefore, the same transgene employed to anchor β-glucuronidase on tumor cells can also enzymatically activate glucuronide imaging agents. Thus, the tumors can first be imaged with a glucuronide probe to ensure proper expression of the β-glucuronidase on tumor cells and then glucuronide prodrugs can be administered to kill the cancer cells.

The novel reporter gene system is designed to combine imaging and gene therapy within one convenient reporter gene system and is compatible with additional reporter gene system. The membrane bound reporter gene systems may also be useful for monitoring gene expression in vivo and in vitro and/or optimizing gene therapy protocols. For example, it was found that a functional mouse β-glucuronidase was stably expressed on the surface of murine CT 26 colon adenocarcinoma tumors where it selectively hydrolyzed its cell impermeable FDGlcU substrate/probe as determined by measuring the levels or intensities of the hydrolyzed fluorescent products. FDGlcU was also converted into a fluorescent product at infected CT26 tumors in live nude mice. The fluorescent intensity in β-glucuronidase-expressing CT26 tumors was about 240 times greater than the fluorescence intensity in control tumors.

As another example, selective imaging of gene expression was also observed after intramural injection of adenoviral β-glucuronidase vector into carcinoma xenografts. The membrane bound β-glucuronidase transgene did not induce any antibody response after hydrodynamic plasmid immunization of Balb/c mice, indicating that the reporter gene product of membrane bound β-glucuronidase displayed low immunogenicity.

A membrane-anchored form of human β-glucuronidase is also shown to allow in vivo gene expression imaging by measuring the levels or intensities of the hydrolyzed fluorescent products of the human β-glucuronidase, demonstrating that surface expression of functional human β-glucuronidase is feasible for gene therapy and imaging. The membrane-bound β-glucuronidase reporter system allows for in vivo non-invasive imaging of gene expression, displays good selectivity with low immunogenicity and may help assess the location, magnitude, and duration of gene expression in living animals and humans.

One embodiment of the invention provides a novel membrane bound reporter gene system to asses the delivery and expression of a gene of interest in living animals. For example, a functional membrane bound enzyme is constructed by anchoring βG to the plasma membrane of cells to allow selective hydrolysis of FDGlcU to a fluorescent reporter in vitro and in vivo. It is shown herein that the membrane bound enzyme does not induce a humoral immune response in mice, suggesting that repeated and persistent imaging of gene expression can be achieved. As another example, proportional expression of the gene of interest and membrane bound reporter gene system can also be optimized and attained by inserting an internal ribosomal entry site or furin-2A-selfprocessing peptide between the genes.

Glucuronides do not readily enter cells due to the presence of a charged carboxylic acid at physiological pH value, thereby preventing contact of glucuronides with lysosomal β-glucuronidase. By anchoring β-glucuronidase on the outer surface of the plasma membrane, maximal activation of glucuronide probes can be achieved to increase imaging sensitivity. Successful development of the membrane bound enzyme system therefore requires efficient transport of membrane bound β-glucuronidase enzyme to the cell surface. TM domain is needed to direct localization onto cell membrane. Spacer domains introduced between β-glucuronidase and the TM allow more flexible assembly of the β-glucuronidase tetramer. As an example, high expression of functional β-glucuronidase on cells is shown herein by creating a chimeric receptor in which β-glucuronidase was fused to the Ig-hinge-like domains of the B7-1 antigen (e-B7) and anchored to the cell surface with the B7-1 TM domain.

Reporter gene products should display low immunogenicity to prevent tissue damage by cellular immune responses and allow repeated and persistent imaging of gene expression. The membrane bound enzyme as described herein can be derived from murine β-glucuronidase, which is shown herein not to induce a detectable antibody response in mice. In addition, prolonged expression of β-glucuronidase in CT26 tumor in vivo suggests that cellular immunity is not induced by the membrane bound murine β-glucuronidase.

Significantly, as another example, a human membrane bound β-glucuronidase is active and allows imaging of gene expression in vivo. These results indicate that this strategy may be extended to human gene therapy. Furthermore, the reporter gene systems herein can be designed to exhibit low immunogenicity in different animals by using β-glucuronidase derived from the species of interest. A range of glucuronide probes and/or many substrates of β-glucuronidase can be used for gene expression imaging with β-glucuronidase membrane bound enzymes.

A membrane bound β-glucuronidase enzyme may allow both imaging and therapy of cancer with the recombinant DNA construct as provided herein since β-glucuronidase has demonstrated antitumor activity in antibody-directed enzyme prodrug therapy (ADEPT) and gene-directed enzyme prodrug therapy (GDEPT). As an example, immunoenzymes formed by conjugating β-glucuronidase to anti-tumor antibodies can selectively activate glucuronide prodrugs, allow accumulation of high drug concentration at the tumor site, produce bystander killing of antigen-negative tumor cells and generate long-lasting protective immunity to subsequent tumor challenge. β-glucuronidase is therefore an attractive enzyme for specific conversion of glucuronide prodrugs for cancer therapy.

Conveniently, the same transgene construct may be employed to assess the specificity and extent of gene transduction in vivo as well as for mediating the glucuronide prodrug therapy of cancer. Real-time detection of glucuronide tracers may also allow estimation of the pharmacokinetics of glucuronide prodrug activation. In addition, the recombinant DNA construct provided herein to be introduced to a host cell is compatible to any existing reporter gene system if introducing of additional recombinant DNA constructs or reporter gene systems into the host cell is desirable.

### EXAMPLES

The membrane reporter transgene systems can be constructed as exemplary membrane bound β-glucuronidase reporter systems by tethering the enzyme β-glucuronidase to a transmembrane domain and/or a spacer domain of an exemplary murine B7 gene. Different exemplary extracellular domains, such as the constant extracellular domain of the mouse B7 gene (e-B7), human γ1 (hinge-CH2-CH3 domains of human IgG1), mutant human γ1 (CH2-CH3 domains of human IgG1) extracellular domain of biliary glycoprotein-1, the extracellular domain of CD44, etc., which are fused to the transmembrane and cytosolic domains of the murine B7 gene were tested by linking the cDNA in-frame to the 3′ end of the human and mouse βG cDNA (the C-terminus of the recombinant β-glucuronidase protein). Linking of exemplary TM domains to 5′ end of β-glucuronidase cDNA (the N-terminus of the recombinant β-glucuronidase protein) were also tested and described in Example 11. Linking a DNA fragment coding for an exemplary GPI anchor at the C-terminus of β-glucuronidase was also tested and is described in Example 12.

Expression of mouse, human and E. coli β-glucuronidase enzymes was found to be located on the cell surface under microscopic examination of Balb/3T3 cells transfected with mouse, human and E. coli membrane bound β-glucuronidase reporter systems with different extracellular spacer domain. After 48 hours of transfection, Balb/3T3 cells were stained with mouse anti-HA mAb and FITC-conjugated goat anti-mouse IgG (Fab′)₂ antibody. Comparison of various constructs of the mouse, human and E. coli membrane bound β-glucuronidase reporter systems, it was found that mouse B7 extracellular domain allowed the most effective surface expression of human and mouse β-glucuronidase enzymes.

The basic techniques for conducting the immunological assays can be found in “Antibodies: A Laboratory Manual”, Harlow and Lane, Cold Spring Harbor Laboratory, Cold Spring Harbor, N.Y. 1989; “Molecular Cloning”, A Laboratory Manual, eds. Sambrook, Fritsch and Maniatis, Cold Spring Harbor Laboratory Press, 1989. and others books and manuals known in the art.

### Example 1

### Recombinant Reporter Gene Systems Having β-Glucuronidase Linked to Mouse B7 Extracellular Domain

The enzymatic activity of β-glucuronidase has been examined by β-glucuronidase microassay in 0.1% BSA/phosphate-buffered saline using recombinant reporter gene constructs having β-glucuronidase (βG) linked to mouse B7 extracellular domain. The mouse B7 extracellular domain allowed high activity of human and mouse β-glucuronidase to be expressed on cells, demonstrating efficient translation, synthesis, and/or proper folding of β-glucuronidase tetramer transported to the outside surface of the cell membranes. Enzyme activities of Balb/3T3 cells that expressed β-glucuronidase on their surface were measured by seeding 1×10⁵ cells/well into 96 wells plates. After 6 hour, the cells were washed one time with phosphate-buffered saline and immediately assayed for β-glucuronidase activity by adding 200 μL 0.1% BSA/phosphate-buffered saline buffer containing 3.2 mM p-nitrophenol β-D-glucuronide at 37° C. for 1 hour. A sample of 150 μL liquid was removed from each well and mixed with 20 μL NaOH in a new 96 well plate. The absorbance of individual wells was measured in an ELISA reader at 405 nm.

### Example 2

### Cell Surface Display of Membrane Bound β-Glucuronidase-B7 Reporter Systems

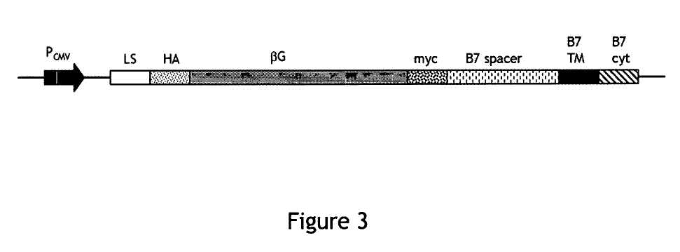

FIG. 3 illustrates an exemplary membrane-bound β-glucuronidase reporter system having cDNA sequences encoding for an immunoglobulin kappa chain leader sequence (LS) followed by an HA epitope (HA), the mature β-glucuronidase (βG) gene, a myc epitope (myc), the immunoglobulin C2-type extracellular region (B7 spacer domain) of B7-1 in addition to the transmembrane (TM) domain and the cytosolic domain of a murine B7-1 gene, where the gene expression is under the control of a promoter, such as a CMV promoter as shown in FIG. 3. Other suitable promoters can also be used.

A DNA fragment of mouse β-glucuronidase cDNA, Seq ID No 3, was fused to the B7 extracellular and transmembrane domains present in a plasmid DNA, p2C11-eB7, and then inserted into the retroviral vector pLNCX (BD Biosciences, San Diego, Calif.) to generate pLNCX-mβG-eB7. The amino acid sequence of the mouse β-glucuronidase for the corresponding mouse β-glucuronidase (βG) cDNA is shown as Seq ID No. 4.

A myc epitope is also present in pLNCX-mβG-eB7. The nucleotide sequence of the myc epitope is shown as Seq ID No. 5. and the corresponding amino acid sequence is shown as Seq ID No. 6. The nucleotide sequence of the extracellular and transmembrane domains of the mouse B7 gene is shown as Seq ID No. 7. The corresponding amino acid sequence of the extracellular and transmembrane domains of the mouse B7 gene is shown as Seq ID No. 8.

The resulting pLNCX-mβG-eB7 plasmid containing a recombinant DNA fragment, which includes a DNA fragment of the mouse β-glucuronidase cDNA, the myc epitope as well as the immunoglobulin-like C2-type extracellular region, transmembrane domain and cytoplasmic tail of the B7-1 antigen. The nucleotide sequence of a mβG-myc-eB7 recombinant DNA fragment is shown as Seq ID No. 9 and the corresponding amino acid sequence is shown as Seq ID No. 10.

A DNA fragment of human β-glucuronidase cDNA, Seq ID No 1, was used to construct pLNCX-hβG-eB7 by replacing the mouse βG cDNA with the human β-glucuronidase cDNA. The corresponding amino acid sequence for the human β-glucuronidase cDNA is shown as Seq ID No. 2.

Recombinant retroviral particles were packaged by co-transfection of pVSVG with pLNCX-mβG-eB7 or pLNCX-hβG-eB7 into GP2-293 cells (Clontech, BD Biosciences, US). After 48 hour, the culture medium was filtered, mixed with 8 μg/ml polybrene and added to CT26 colon carcinoma cells or EJ human bladder carcinoma cells, respectively. The cells were selected in G418 and sorted on a flow cytometer to generate CT26/mβG or EJ/hβG cells.

As exemplified in FIG. 3, a retrovirus vector, pLNCX-mβG-eB7, (murine β-glucuronidase fused to the immunoglobulin-like C2-type extracellular region, transmembrane domain and cytoplasmic tail of the murine B7-1 antigen) can be used to direct the expression of mouse β-glucuronidase to the plasma membrane of mammalian cells. Any mammalian cell expression vector, such as retroviral, adenoviral, adeno-associated vectors, herpes simplex vectors, etc can also be used. CT26 murine colon adenocarcinoma cells were infected with recombinant retroviral particles and selected in G418 to obtain CT26/mβG-eB7 cells. The cells were immunofluorescence stained for the presence of the myc epitope in cells infected with pLNCX-mβG-eB7. The cells were also incubated with fluorescein di-β-D-glucuronide (FDGlcU) probes as substrates for membrane bound murine β-glucuronidase enzyme to assay for β-glucuronidase enzymatic activity.

CT26 murine colon carcinoma cells, EJ human bladder carcinoma cells, HCC36 hepatocellular carcinoma cells, 293N adenovirus packaging cells and GP2-293 retrovirus packaging cells were grown in Dulbecco's Minimal Essential Medium (Sigma, St Louis, Mo., USA) supplemented with 10% heat-inactivated bovine calf serum, 100 units/ml penicillin and 100 μg/ml streptomycin at 37° C. in an atmosphere of 5% CO₂.

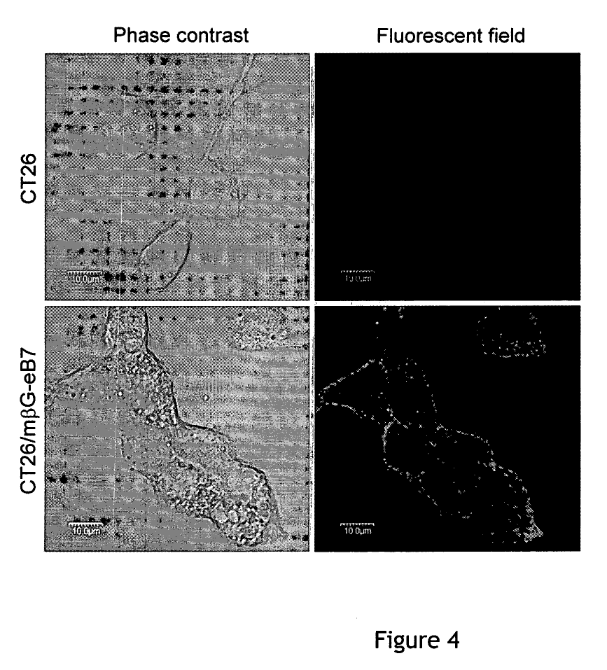

FIG. 4 shows cells surface display of an exemplary functional mouse membrane bound β-glucuronidase reporter system expressed in live CT26 cells. For example, functional expression of membrane bound β-glucuronidase on a host cell can be measured by incubating the host cells (with and without the recombinant reporter gene systems, such as CT26 or CT26/mβG-eB7 cells) with 40 μM FDGlcU (Invitrogen, Calsbad, Calif.) in phosphate-buffered saline containing 0.1% BSA, pH 6.5 at 37° C. for 40 minutes and then staining the cells with anti-myc antibody (5 μg/ml, clone Myc 1-9EIO.2, American Type Culture Collection, Manassas, Va.) at about 4° C. for about 1 hour. The cells can be washed with cold phosphate-buffered saline and incubated with rhodamine-conjugated rabbit anti-mouse IgG antibody (5 μg/ml) at about 4° C. for about 1 hour. The cells are then washed with cold phosphate-buffered saline, mounted with fluorescence mounting medium (anti fade; DakoCytomation, Carpinteria, Calif.), and viewed under a digital fluorescence confocal microscope.

As shown in FIG. 4, CT26 cells with and without murine β-glucuronidase (mβG-e-B7) transgene were immunofluorescence stained for the presence of the myc epitope in mβG-e-B7 (red) and then incubated with FDGlcU probe (green) before observation under a digital fluorescence confocal microscopy system. As shown in FIG. 4, only CT26/mβG-e-B7 cells, showing as red and green, express high levels of membrane bound mβG-e-B7 on their membrane surface for converting FDGlcU to fluorescein (green). The parental CT26 cells did not express the myc-epitope and no conversion of non-fluorescent FDGlcU to fluorescein was observed, demonstrating that membrane bound murine βG was functionally active on the cell surface.

### Example 3

### In Vivo Imaging of Membrane Bound β-Glucuronidase-B7 Reporter Systems

To investigate whether expression sites of membrane bound β-glucuronidase reporter system could be non-invasively detected (in vivo imaging), Balb/c mice bearing established CT26 and CT26/mβG-eB7 colon tumors in their left and right chest regions, respectively, were intravenously injected with about 500 μg of non-fluorescent FDGlcU. Whole-body images of the mice were acquired by performing 3 minute scans.

As an example, Balb/c mice (n=3) bearing established CT26 and CT26/mβG tumors (200-300 mm³) in their left and right chest regions, respectively, were i.v. injected with 500 μg FDGlcU. Whole-body images of pentobarbital-anesthetized mice were obtained by performing 3 minutes scans over 90 minutes on a Kodak IS2000MM optical imaging system. The fluorescence intensities were analyzed with KODAK 1D Image Analysis Software.

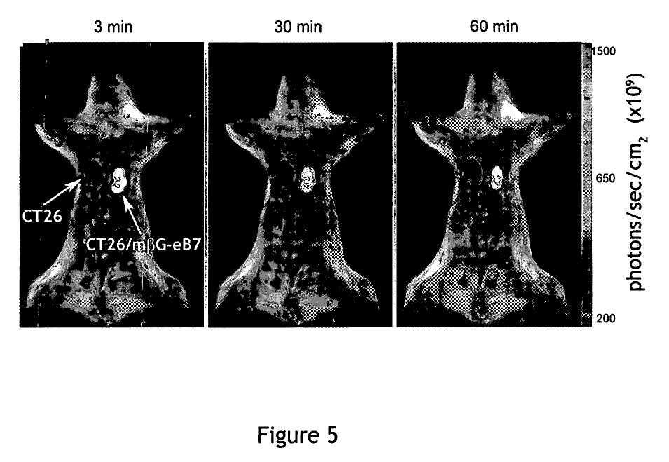

FIG. 5 shows the results of in vivo imaging of an exemplary functional mouse membrane bound β-glucuronidase reporter system using FDGlcU as its substrate, which is intravenously injected into mice bearing CT26 tumors (left) and CT26/mβG-eB7 (right) tumors with whole-body images acquired at the indicated times of 3 mins, 30 mins, 60 mins, respectively. FDGlcU is shown to be selectively converted to fluorescein at the sites of membrane bound β-glucuronidase expression in CT26/mβG tumors but not in the control CT26 tumors.

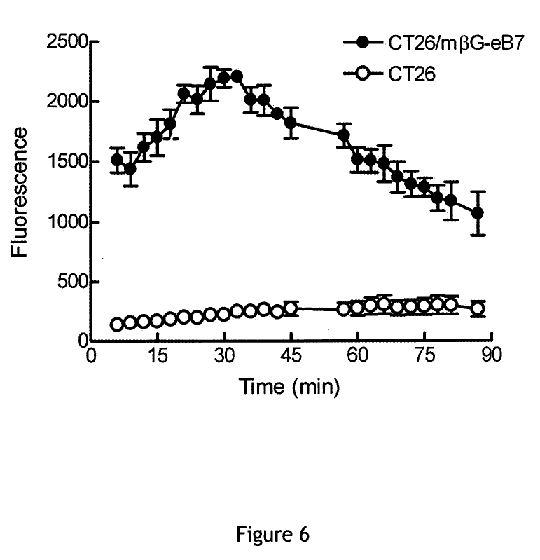

FIG. 6 shows the pharmacokinetic results of FDGlcU activation using the exemplary system of FIG. 5. The distribution of activated FDGlcU in CT26 (open circle) and CT26/mβG-eB7 (black dot) tumors (n=4) was determined by measuring fluorescence intensities in 3 minute scans performed over 90 minutes. Serial imaging analysis showed that the highest fluorescence was observed at 30 min after FDGlcU injection.

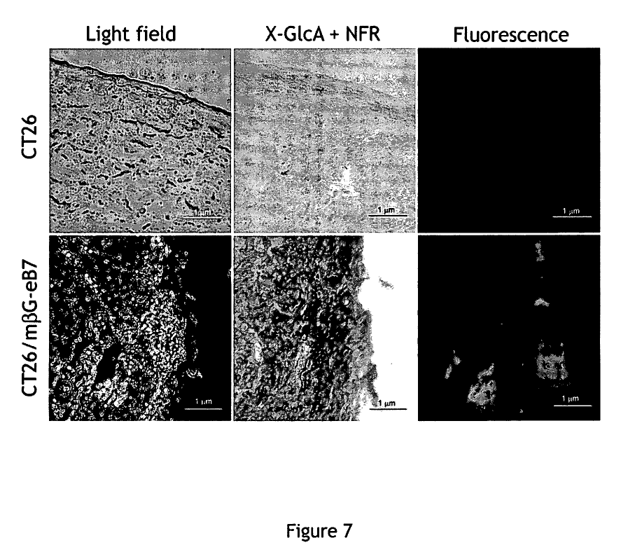

FIG. 7 shows the results of staining thin cell sections of CT26 (upper panels) and CT26/mβG-eB7 (lower panels) tumors stained with X-GlcA and nuclear fast red (NFR). To verify the imaging results, the tumors were resected and adjacent tumor sections were examined under a fluorescence microscope or stained with X-GlcA to examine the functional expression of the membrane bound β-glucuronidase (βG). Adjacent sections were viewed under phase contrast or fluorescence microscopes. For example, tumors were excised at 30 minutes after FDGlcU injection, and then the excised tumors were embedded in Tissue-Tek OCT in liquid nitrogen, and sectioned into 10 μm slices. Adjacent tumor sections were stained for β-glucuronidase activity with the β-Glucuronidase Reporter Gene Staining Kit (Sigma) and counterstained with nuclear fast red. The sections were examined on an upright microscope (Olympus BX41) or viewed in phase contrast and fluorescence modes on an inverted epifluorescence microscope (Zeiss Axiovert). As shown in FIG. 7, the regions of active fluorescein displayed concomitant blue X-GlcA staining at CT26/mβG-eB7 tumor sections (lower panel) but not at control CT26 tumor sections (upper panel), consistent with selective hydrolysis of FDGlcU at sites of membrane bound β-glucuronidase (βG) expression in vivo.

### Example 4

### Biodistribution of Active Substrates of the Membrane Bound β-Glucuronidase (βG) In Vivo

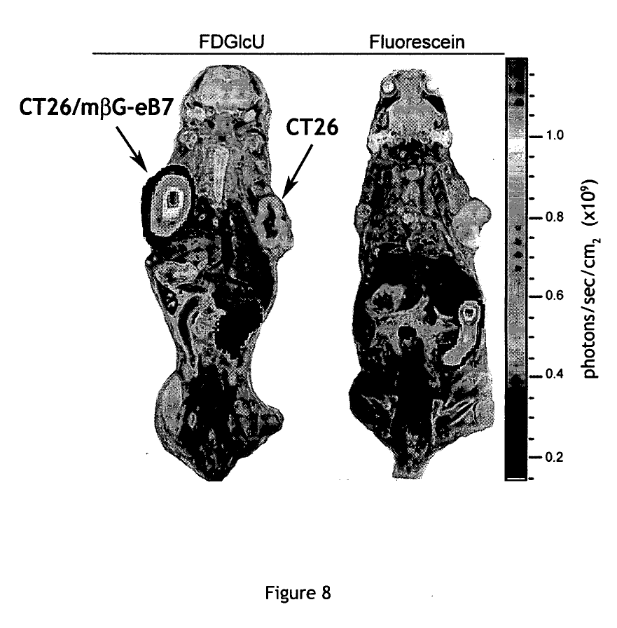

FIG. 8 shows the results of in vivo biodistribution of fluorescein in mice according to one embodiment of the invention. In vivo biodistribution of fluorescein was measured around 30 minutes after i.v. injection of the substrate, FDGlcU, (left panel) or fluorescein (right panel). The biodistribution of activated FDGlcU was examined by killing tumor-bearing mice 30 minutes after they received an injection of FDGlcU or fluorescein and then performing optical imaging of whole-body frozen sections. The intensity of the fluorescence of whole-body sections was measured with the IVIS® Imaging System 50 (Xenogen, Alameda, Calif.).

For example, mice (n=3) were i.v. injected with 500 μg FDGlcU or fluorescein 30 min before the mice were dipped into isopentane at liquid nitrogen temperatures and embedded on a cryostat holder (7×5 cm) in 4% carboxylmethylcellulose. The fluorescence signals of 30 μm whole-body sections were measured on a IVIS® Imaging System 50 (Xenogen, Alameda, Calif.) and the regions of interest were analyzed with Living Image® software (Xenogen).

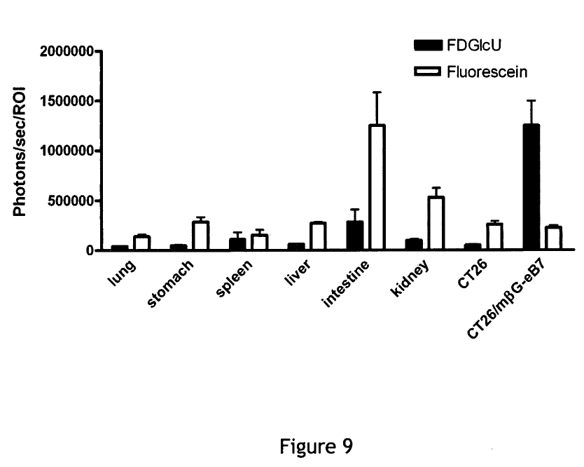

FIG. 9 is a graph showing a comparison of the results of the example of FIG. 8 in different organ regions of interest according to one embodiment of the invention. After i.v. injection of FDGlcU or fluorescein, mice were analyzed with Living Image® software. FDGlcU was selectively converted to fluorescein in CT26/mβG-eB7 tumors but not in CT26 tumors (FIG. 8, left panel). The fluorescent intensity in CT26/mβG-eB7 tumors was about 240 times greater than in the CT26 tumors (FIG. 9). Fluorescent signals were also observed in the intestinal tract. In contrast to the biodistribution of activated FDGlcU (FIG. 8, right panel), intravenously administered fluorescein largely accumulated in the intestinal tract and kidneys.

### Example 5

### Infection of Tumor Cell Lines by Membrane Bound β-Glucuronidase (βG) Reporter Systems

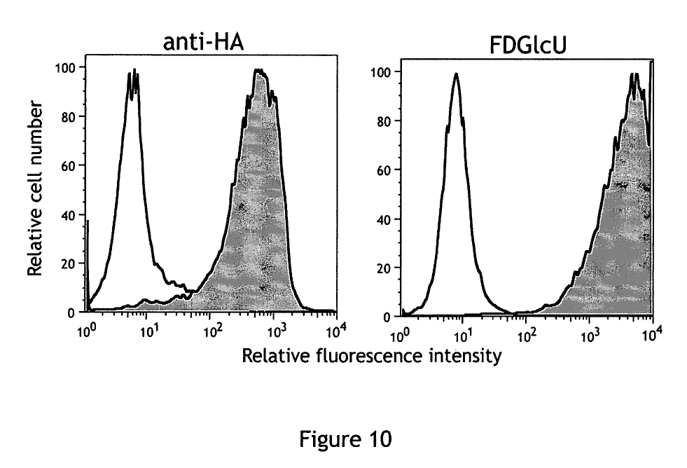

FIG. 10 shows the results of exemplary HCC36 tumor cells (open curve) and HCC36 cells infected with Ad5/mβG-eB7 (solid curve) to examine whether the expression of the membrane bound β-glucuronidase enzyme could be detected after adenoviral-mediated infection of HCC36 human hepatocellular carcinoma cells. HCC36 cells (open curve) and HCC36 cells infected with Ad5/mβG-eB7 (solid curve) were stained for the presence of the HA epitope in mβG-eB7 (left panel) or directly stained with FDGlcU (right panel) and then analyzed on a flow cytometer.

To image Ad5/mβG-eB7, nude mice (n=3) bearing established HCC36 tumors (200-300 mm³) in their left and right chest's regions were injected in the right HCC36 tumor with 10⁹ pfu of Ad5/mβG-eB7 in 50 μl of phosphate-buffered saline (PBS). Two days later, the mice were i.v. injected with 500 μg of FDGlcU. Whole-body images were obtained in 3 min scans over 2 hour.

As shown by staining of membrane β-glucuronidase with anti-myc antibody in the left panel of FIG. 10, HCC36 cells were successfully infected with Ad5/mβG-eB7. In addition, as shown in the right panel of FIG. 10, FDGlcU is activated to fluorescent product at the infected cells.

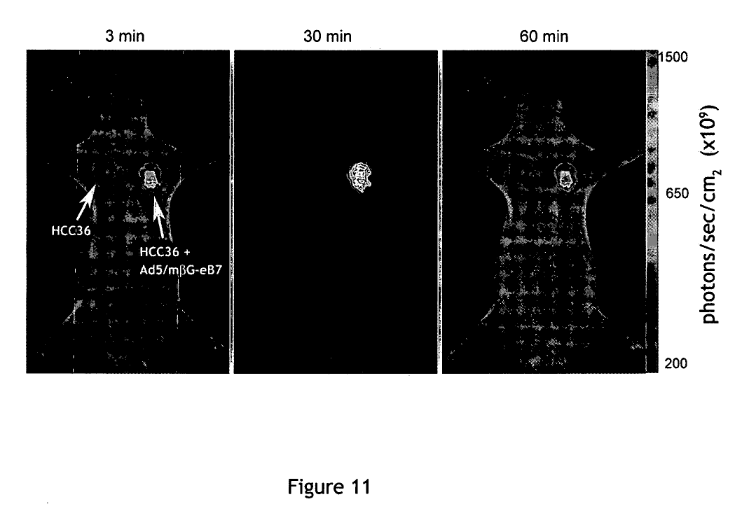

FIG. 11 shows the example in which a nude mouse was injected in the left and right side of the chest with HCC36 tumor cells. After the tumors grew, 10⁹ pfu of Ad5/mβG-eB7 was directly injected into the HCC36 tumor on the right side of the mouse. Two days later, the mouse was i.v. injected with FDGlcU and whole-body optical imaging was performed. As shown in FIG. 11, the HCC36 tumor that was injected with Ad5/mβG-eB7 displayed obvious fluorescence as compared with non-infected HCC36 tumors.

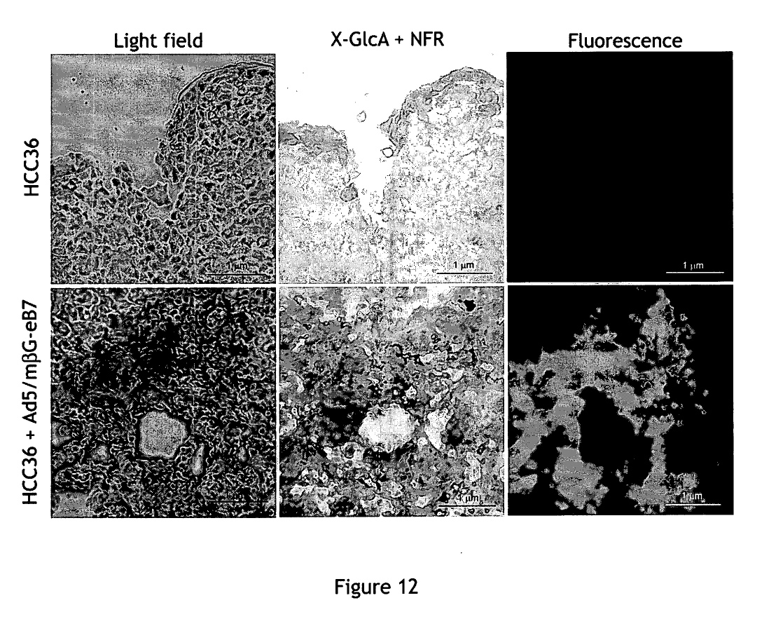

FIG. 12 shows the results of histological staining of cell sections of HCC36 tumors (upper panel) and Ad5/mβG-eB7 injected HCC36 tumors (lower panel). Tumor cells were collected 30 minutes after FDGlcU injection were stained with X-GlcA and nuclear fast red and then viewed under phase contrast and fluorescence microscopes. Histological staining for β-glucuronidase activity revealed strong fluorescence in tumor sections obtained from Ad5/βG infected HCC36 tumors (FIG. 12, lower panel) but not in sections obtained from non-infected tumors (FIG. 12, upper panel). These results show that FDGlcU substrate/probe could specifically locate sites of adenoviral-mediated gene expression in vivo.

### Example 6

### Immunogenicity of Membrane Bound β-Glucuronidase (βG) Reporter Systems

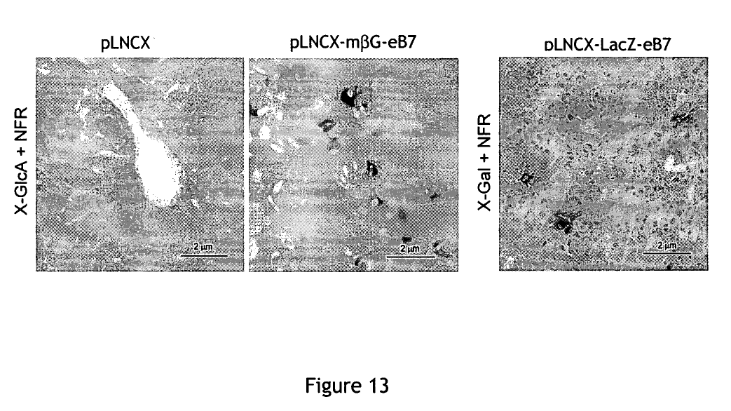

FIG. 13 shows low immunogenicity of an exemplary functional mouse membrane bound β-glucuronidase reporter system as compared to high immunogenicity of a LacZ membrane bound reporter system. To test as an useful expression vector for gene therapy, the immunogenicity of the membrane bound β-glucuronidase reporter system was examined after hydrodynamic-based gene transfer of pLNCX-mβG-eB7 into Balb/c mice. Control groups of mice were injected with pLNCX vector or pLNCX-LacZ-eB7, which encodes a membrane form of E. coli β-galactosidase. Mouse livers were excised 2 days after hydrodynamic-based injection of pLNCX, pLNCX-mβG-eB7 or pLNCX-LacZ-eB7 plasmids. Frozen liver sections were stained for membrane β-glucuronidase activity or LacZ activity and then counterstained with nuclear fast red (NFR). Serum samples were collected and livers were excised, embedded in Tissue-Tek OCT and cut into sections for X-GlcA or X-Gal staining to detect for functional expression of membrane β-glucuronidase or LacZ. As shown in FIG. 13, mβG-eB7 and LacZ-eB7 were expressed in the liver as determined by specific hydrolysis of X-GlcA or X-Gal. For example, groups of about 4 to 6 Balb/c mice can be anesthetized by pentobarbital (65 mg/kg), and then injected with 10 μg pLNCX (negative control), pLNCX-mβG-eB7, or pLNCX-LacZ-eB7 (positive control), a vector containing the LacZ gene fused to the eB7 domain to anchor E. coli β-galactosidase on the cell surface. The plasmids were i.v. injected in 2 ml phosphate-buffered saline within 8 seconds for hydrodynamic-based gene transfer on days 1 and 8.

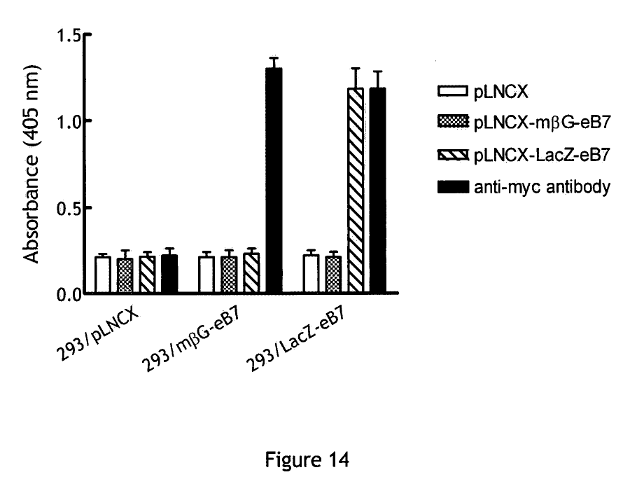

FIG. 14 shows the results of testing serum samples to demonstrate low immunogenicity of an exemplary functional mouse membrane bound β-glucuronidase (βG) reporter system as compared to high immunogenicity of a LacZ membrane bound reporter system. The humoral immune response against the membrane bound βG was examined by detecting antibody binding to 293 cells that were transiently transfected with pLNCX, pLNCX-mβG-eB7 or pLNCX-LacZ-eB7.

Serum samples collected from Balb/c mice 10 days after hydrodynamic-based gene transduction were assayed by ELISA for the presence of antibodies against 293 cells that were transiently transfected with pLNCX, pLNCX-mβG-eB7 or pLNCX-LacZ-eB7. The binding of an anti-myc antibody to the myc epitope present in the surface enzymes was also assayed (black bars). Serum samples were collected 10 days after the second injection. Pre-immune and immune serum samples (diluted 1:250 in phosphate-buffered saline) were added to the 96 wells plates coated with 293 cells that were transiently transfected with pLNCX, pLNCX-mβG-eB7, or pLNCX-LacZ-eB7 plasmids.

Binding of the serum and anti-myc antibodies to the cells was detected by serial addition of horse-radish peroxidase conjugated goat anti-mouse antibody (2 μg/ml) and 100 μl/well ABTS substrate [0.4 mg/ml of 2,2′-azino-di(3-ethylbenzthiazoline-6-sulfonic acid), 0.003% H₂O₂, 100 mM phosphate-citrate, pH 4.0] for 30 min at room temperature. The absorbance (405 nm) of the wells was measured in a microplate reader (Molecular Device, Menlo Park, Calif.). To assess gene expression, livers were excised at about 48 hour after hydrodynamic-based injection of plasmids, embedded in Tissue-Tek OCT in liquid nitrogen and cut into 10 μm sections.

Liver sections were stained for βG activity by using the β-Glucuronidase Reporter Gene Staining Kit (Sigma) or β-Galactosidase activity with the β-Gal Staining Kit (Invitrogen) according to the manufacturer's instructions. All sections were examined on an upright microscope (Olympus BX41, Japan). Results show the mean absorbance values of triplicate determinations. Bars, SE. In general, statistical significance of differences between mean values was estimated with Excel (Microsoft, Redmond, W A) using the independent t-test for unequal variances. P values of less than 0.05 were considered statistically significant.

The presence of membrane bound mouse β-glucuronidase enzyme on 293 cells was indicated by binding to anti-myc antibody (black bars). Antibody titers were detected in mice injected with pLNCX-LacZ-eB7 (striped bar), indicating that β-galactosidase was immunogenic. By contrast, no specific antibody titer was detected in mice injected with pLNCX-mβG-eB7 transfected 293 cells. In addition, prolonged growth of CT26/mβG-eB7 tumors in Balb/c mice suggests that cellular immunity was not induced by mβG-eB7. Thus, the mβG memzyme can be stably expressed on cells in an active form and did not induce any specific immune response, prerequisites for repetitive and persistent imaging in live animals.

### Example 7

### Non-Invasive Imaging of Membrane Bound β-Glucuronidase Reporter Systems

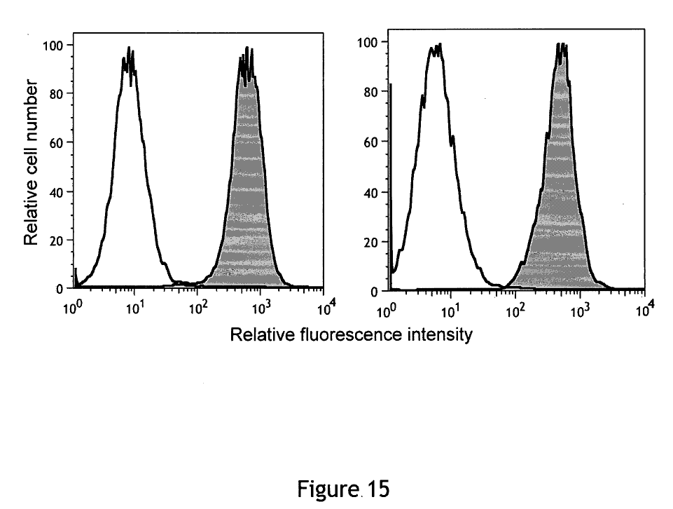

FIG. 15 shows the analysis and imaging of an exemplary functional human membrane bound β-glucuronidase reporter system to investigate whether expression of membrane bound human β-glucuronidase could be active on cell surface and allow non-invasive imaging by FDGlcU. Retroviral vector pLNCX-hβG-eB7 was constructed and infected to EJ human bladder carcinoma cells (EJ/hβG-eB7) to express functionally active human β-glucuronidase on the membrane. hβG-eB7 was imaged in nude mice (n=3) bearing established EJ and EJ/hβG-eB7 tumors (200-300 mm³) in their left and right chest regions, respectively, by i.v. injecting 500 μg FDGlcU and performing imaging and histological analysis of fluorescent intensity and βG activity as described above.

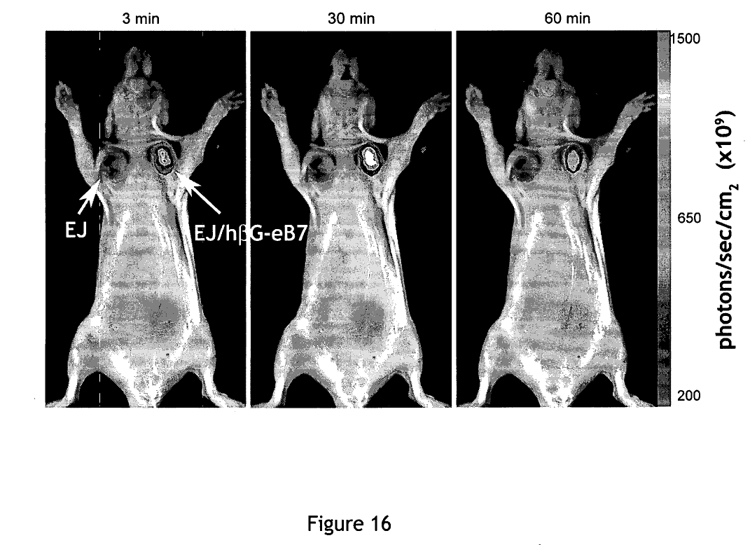

FIG. 16 shows the results of the analysis and imaging of an exemplary functional human membrane bound β-glucuronidase reporter system i.v. injected into nude mice capable of infecting mouse cells and developing tumors. FDGlcU was i.v. injected into mice bearing EJ (left) and EJ/hβG-eB7 (right) tumors and whole-body images were acquired at the indicated times (3 mins, 30 mins, 60 mins). FDGlcU was preferentially converted to a fluorescent reporter in EJ/hβG-eB7 tumors but not in control tumors in mice as determined by optical imaging, indicating that human β-glucuronidase can act as reporter gene for noninvasive imaging of gene expression in vivo. Live EJ cells (open curve) and EJ/hβG-eB7 cells (solid curve) were immunofluorescence stained for the myc epitope (left panel) or incubated with FDGlcU (right panel), respectively, and analyzed on a flow cytometer.

### Example 8

### Additional Examples for the Transmembrane Domains and/or Spacer Domains of the Membrane Bound β-Glucuronidase Reporter Systems and the Influence of Transmembrane Domains and Spacer Domains on β-Glucuronidase Surface Expression

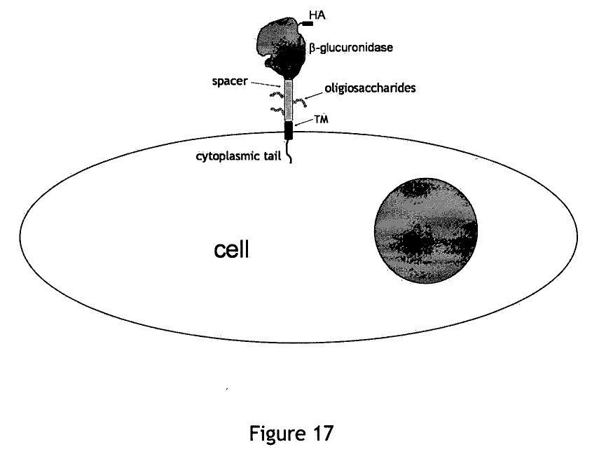

The effectiveness of anchoring β-glucuronidase on cells with different juxtamembrane spacer domains and transmembrane domains were examined. FIG. 17 shows another exemplary membrane bound β-glucuronidase reporter systems. Various spacer domains and transmembrane domains were tested herein. In addition, because different sources of β-glucuronidase enzymes display unique kinetic properties, surface expression and surface activity of at least three β-glucuronidase, E. coli β-glucuronidase (eβG), murine β-glucuronidase (mβG), and human β-glucuronidase (hβG), were examined and compared.

The transmembrane domain (TM) and the juxtamembrane “spacer” domain can affect the expression levels and activity of a membrane bound β-glucuronidase. A panel of human β-glucuronidase transgenes was constructed in which an immunoglobulin chain signal sequence and an HA tag is fused in frame to the 5′ end of the mature human β-glucuronidase cDNA.

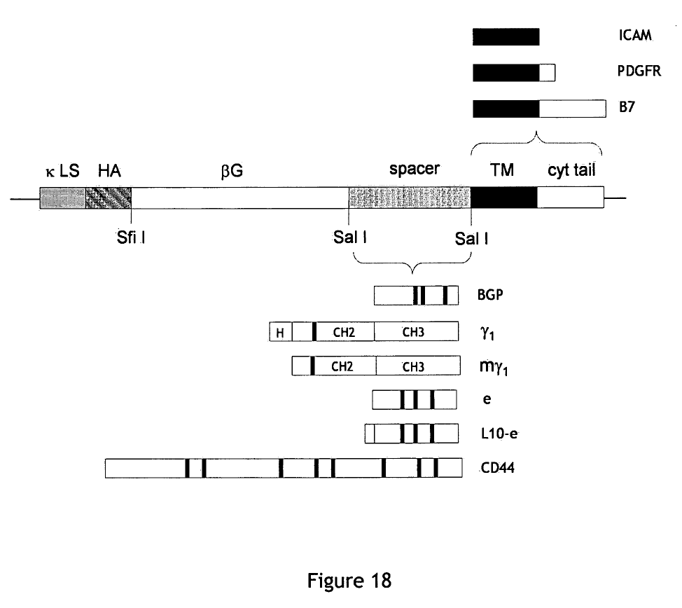

FIG. 18 illustrates various TM domains derived from the human platelet-derived growth factor receptor (PDGFR), the murine B7-1 antigen (B7) and human intracellular adhesion molecule 1 (ICAM-1) and various spacer domains derived from an immunoglobulin-like V-type domain (domain 1) of human biliary glycoprotein I (BGP), hinge-CH₂—CH₃ domains of human IgG₁ (γ₁), CH₂—CH₃ domains of human IgG₁ (hβG-mγ₁-B7), a Ig-like C2-type and Ig-hinge-like domain of the murine B7-1 antigen (e), and the extracellular portion of human CD44E (CD44), which are employed to anchor human β-glucuronidase to on the cell surface.

As shown in FIG. 18, these βG recombinant constructs may include an immunoglobulin kappa chain leader sequence (κLS) followed by an HA epitope (HA) and the full-length human βG cDNA. Black bars indicate the presence of N-linked glycosylation sites. TM domains were derived from human ICAM-1, the human platelet-derived growth factor receptor or the mouse B7-1 antigen. In addition to the TM domains, the cDNA fragment for the TM domain of PDGFR TM also includes six amino acids of its cytoplasmic tail, whereas the cDNA fragment for the TM domain of B7 TM includes the entire 38 amino acid cytoplasmic tail of the B7-1 antigen.

The various spacer domains as listed in FIG. 18 can be introduced between a human β-glucuronidase cDNA and a DNA fragment encoding a B7 TM domain. The resulting recombinant DNA constructs were obtained and included hβG-immunoglobulin-like V-type domain I of human biliary glycoprotein I (hβG-BGP-B7), hβG-hinge-CH₂—CH₃ domains of human IgG₁ (hβG-γ₁-B7), hβG-CH₂—CH₃ domains of human IgG₁ (hβG-mγ₁-B7), hβG-1 g-like C2-type and Ig-hinge-like domains of the murine B7-1 antigen (hβG-e-B7), and hβG-extracellular domains of human CD44E (hβG-CD44-B7). The γ₁ domain allows formation of disulfide linked dimers.

In addition to human β-glucuronidase (hβG) recombinant constructs, the various spacer domains listed in FIG. 18 can be introduced between a DNA fragment encoding a TM domain and a second DNA fragment encoding an E. coli β-glucuronidase (eβG) or murine β-glucuronidase (mβG). In addition, a linker domain can be introduced. For example, a DNA fragment coding a 10 amino acid flexible linker (GGGGSGGGGS) was appended to the 3′ end of hβG in hβG-L-e-B7 in order to examine the influence of additional flexibility between the domains.

In general, insertion of the hβG-e-B7, mβG-e-B7 and βG-e-B7 transgenes into the retroviral vector pLNCX (BD Biosciences, San Diego, Calif.) generated the retroviral vectors pLNCX-hβG-e-B7, pLNCX-mβG-e-B7 and pLNCX-eβG-e-B7. Expression of these transgenes/recombinant DNA constructs was under control of the CMV promoter. The hβG cDNA fragment in pLNCX-hβG, the mβG cDNA fragment in pLHCX-mβG and the eβG cDNA fragment in pGUS N358S (Clontech, Mountain View, Calif.) were inserted into p2C11-PDGFR, p2C11-BGP-B7, p2C11-e-B7, p2C11-γ₁-B7, and p2C11-CD44-B7 to replace the 2C11 scFv gene and create various recombinant DNA constructs for surface expression of hβG, mβG and eβG with various spacer and TM domains. Removal of the immunoglobulin hinge region in the γ₁ domain of p-hβG-γ1-B7 produced p-hβG-mγ₁-B7. p-hβG-ICAM was generated by inserting the hβG cDNA fragment between the ICAM-1 leader sequence and ICAM-1 transmembrane domain in pLTM-1 (generously provided by Dr. Alister Craig, University of Oxford, Great Britain). In addition, p-hβG-L-e-B7 includes cDNA for a 10 amino-acid linker (GGGGSGGGGS) at the 5′-end of the e-B7 domain.

These β-glucuronidase transgene/recombinant constructs were transiently transfected into murine 3T3 fibroblasts and other cell lines. The protein products were detected by immunoblotting whole cell lysates with an antibody against the HA epitope present in the resulting chimeric recombinant proteins. In general, Balb/3T3 fibroblasts (CCL-163) and CT26 murine colon carcinoma cells (CRL-2638) were grown in DMEM (high glucose) supplemented with about 10% of bovine serum, about 2.98 g/L of HEPES buffer, about 2 g/L of NaHCO₃, about 100 U/mL of penicillin, and about 100 μg/mL of streptomycin. EJ human bladder carcinoma cells were cultured in RPMI containing the same supplements. The cells were free of mycoplasma as determined by PCR (polymerase chain reaction). For example, 3T3 fibroblast cells were transfected with plasmid DNA using Lipofectamine 2000 (Gibco Laboratories, Grand Island, N.Y.). In addition, to generate stable cell lines, pLNCX-eβG-e-B7, pLNCX-hβG-e-B7 and pLNCX-mβG-e-B7 were cotransfected with pVSVG in GP293 cells (Clontech) to produce recombinant retroviral particles. Two days after transfection, the culture medium was filtered, mixed with about 8 μg/ml of polybrene and added to EJ cells or CT26 cells. Stable cell lines were selected in medium containing G418.

Western blot analysis was performed according to standard procedures. For example, transiently-transfected 3T3 fibroblasts were boiled in reducing SDS buffer, electrophoresed on a SDS-PAGE and transferred to PVDF membranes. Membranes were sequentially probed with anti-HA antibody or mAb 1B3 against human β-glucuronidase followed by HRP-conjugated secondary antibody. The membranes were stripped and re-probed with anti-β-actin antibody. Bands were visualized by ECL detection (Pierce, Rockford, Ill.). Relative expression levels of β-glucuronidase were normalized to β-actin band intensities using the shareware program NIH Image (http://rsb.info.nih.gov/nih-image/download.html).

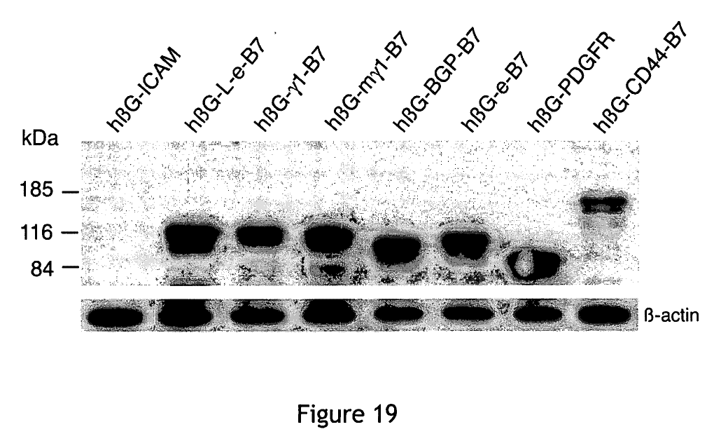

FIG. 19 illustrates immunoblots of exemplary membrane-bound β-glucuronidase reporter systems expressed in 3T3 fibroblast cells. Cell lysates prepared from 3T3 fibroblasts that were transiently transfected with transgenes coding for the indicated chimeric enzymes were immunoblotted with anti-HA antibody (upper panel) or anti-β-actin antibody (lower panel). Transfected cell lysates were immunoblotted with anti-hβG rabbit serum (detecting human β-glucuronidase) or anti-β-actin antibody (as a control). As shown in FIG. 19, the transfected fibroblasts expressed various chimeric β-glucuronidase recombinant proteins at similar levels.

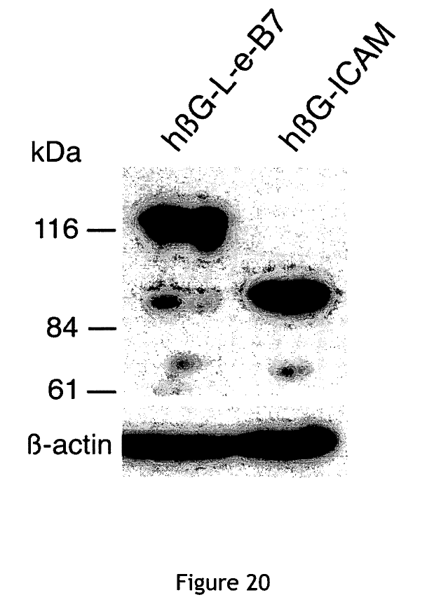

FIG. 20 illustrates immunoblots of exemplary membrane-bound β-glucuronidase reporter systems expressed in 3T3 fibroblast cells according to another embodiment of the invention. Cell lysates prepared from 3T3 fibroblasts that were transiently transfected with transgenes coding for the indicated chimeric enzymes were immunoblotted with anti-human β-glucuronidase antiserum (upper panel) or anti-β-actin antibody (lower panel). hβG-ICAM does not contain the HA epitope so is not detected in FIG. 19 but is detected with anti-human β-glucuronidase antiserum in FIG. 20.

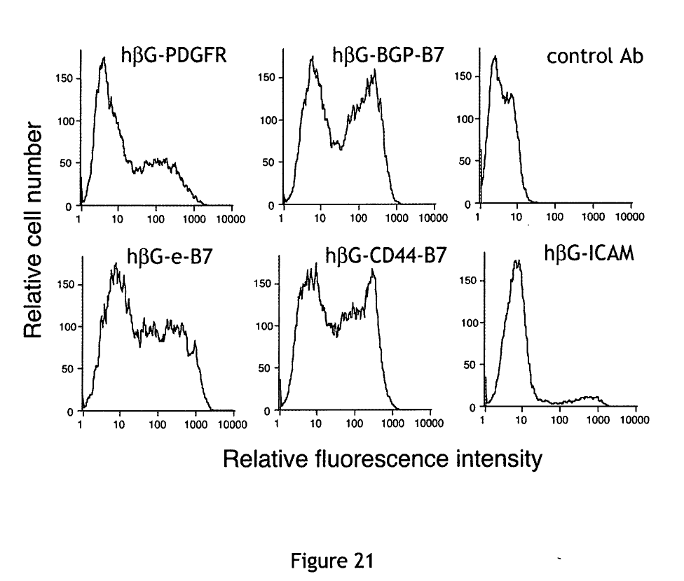

FIG. 21 shows the immunofluorescence of various exemplary functional membrane-bound β-glucuronidase reporter systems in 3T3 fibroblast cells according to embodiments of the invention. Live transiently transfected fibroblasts were immunofluorescence stained for human β-glucuronidase and analyzed on a flow cytometer. As shown in FIG. 21, immunofluorescence staining of transiently transfected fibroblasts shows that hβG-ICAM is poorly expressed in transfected 3T3 cells, hβG-PDGFR is moderately expressed, and hβG-BGP-B7, hβG-e-B7 and hβG-CD44-B7 are highly expressed.

For performing flow cytometer analysis, transfected cells were stained by incubating the cells with an anti-HA antibody followed by a FITC-conjugated goat anti-rat F(ab′)₂ fragment. Alternatively, the cells can be stained with mAb 1E8 against E. coli β-glucuronidase, mAb 7G8 against human β-glucuronidase or mAb 7G7 against murine β-glucuronidase followed by the appropriate secondary FITC-labeled secondary antibody. The surface immunofluorescence of 10,000 viable cells was measured with a FACS caliber flow cytometer (Becton Dickinson, Mountain View, Calif.) and fluorescence intensities were analyzed with Flowjo V3.2 (Tree Star, Inc., San Carlos, Calif.).

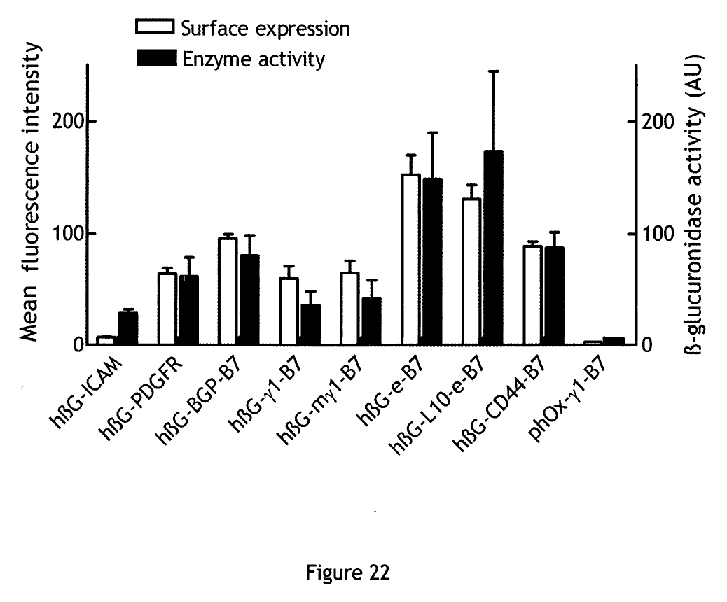

FIG. 22 shows comparison of human β-glucuronidase expression on cells and β-glucuronidase enzyme activity. Membrane bound human β-glucuronidase recombinant constructs were transiently transfected in 3T3 fibroblast cells to express human β-glucuronidase on the cells according to embodiments of the invention. After 2 days following transfection, the cells were immunofluorescence stained for surface β-glucuronidase expression and analyzed on a flow cytometer (left axis) or directly assayed for β-glucuronidase activity (right axis). Results represent mean values of three determinations. Bars, SE.

For performing surface enzyme activity assay, transiently-transfected 3T3 fibroblasts in 96-well microplates were washed once with phosphate-buffered saline and immediately assayed for βG activity by adding about 200 μl of phosphate-buffered saline (pH 7.0) buffer containing 0.1% of BSA and 0.25 mM of a β-glucuronidase substrate, 4-methylumbelliferyl β-D-glucuronide, for 30 min at 37° C. About 150 μl of the resulting mixture was transferred to a 96-well fluorescence microplate and mixed with about 75 μl of a stop buffer containing about 1 M of glycine and about 0.5 M of sodium bicarbonate at pH 11. The fluorescence of each mixture was measured at an excitation wavelength of 365 nm and an emission wavelength of 455 nm.

As shown in FIG. 22, the recombinant hβG-e-B7 and hβG-L-e-B7 constructs produce the highest levels of human β-glucuronidase expression in 3T3 cells (open bars). Comparatively, the recombinant hβG-e-B7 and hβG-L-e-B7 constructs also display the greatest levels of β-glucuronidase enzyme activities on the cell surfaces (solid bars).

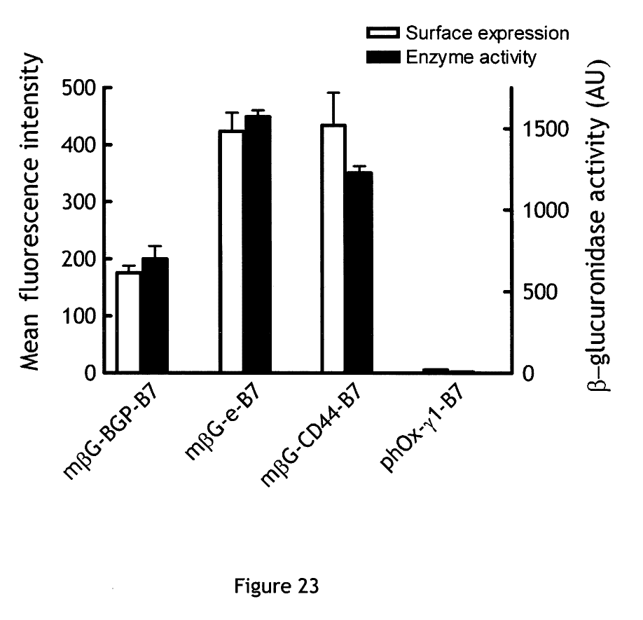

FIG. 23 shows the results of surface immunofluorescence or β-glucuronidase enzyme activity of membrane bound mouse β-glucuronidase recombinant constructs expressed in transiently transfected 3T3 fibroblast cells according to embodiments of the invention. Comparison of mouse β-glucuronidase expression levels on fibroblasts show that the recombinant mβG-e-B7 and mβG-CD44-B7 constructs direct about two fold higher levels of membrane bound mouse β-glucuronidase to the cell surfaces as compared to the mβG-BGP-B7 construct. In addition, a plasmid, phOx-γ1-B7, which contains a highly expressed scFv protein domain, was not expressed and was included to demonstrate the specificity of the anti-β-glucuronidase antibody and enzyme activity assays.

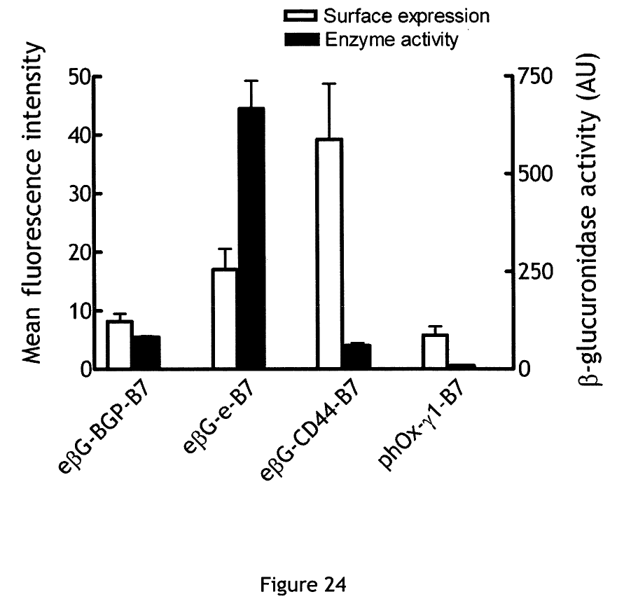

FIG. 24 shows the results of surface immunofluorescence or β-glucuronidase enzyme activity of membrane bound E. coli β-glucuronidase recombinant constructs expressed in 3T3 fibroblast cells according to embodiments of the invention. Although the expression of E. coli β-glucuronidase was low regardless of the spacer domains used, the recombinant eβG-CD44-B7 construct allowed about twice the levels of E. coli β-glucuronidase expression on the cell surface as compared to the recombinant eβG-eB7 construct.

The enzymatic activity of eβG-CD44-B7, however, was dramatically lower than the enzymatic activity of eβG-e-B7, indicating that the CD44 spacer domain did not allow proper folding or formation of the E. coli β-glucuronidase tetramer. Taken together, our results demonstrate that the B7-1 spacer domain and the B7 transmembrane allow the highest levels of active β-glucuronidase expression in vivo and in vitro.

### Example 9

### Stable Expression of Mouse β-Glucuronidase, Human β-Glucuronidase and E. coli β-Glucuronidase on EJ Carcinoma Cells

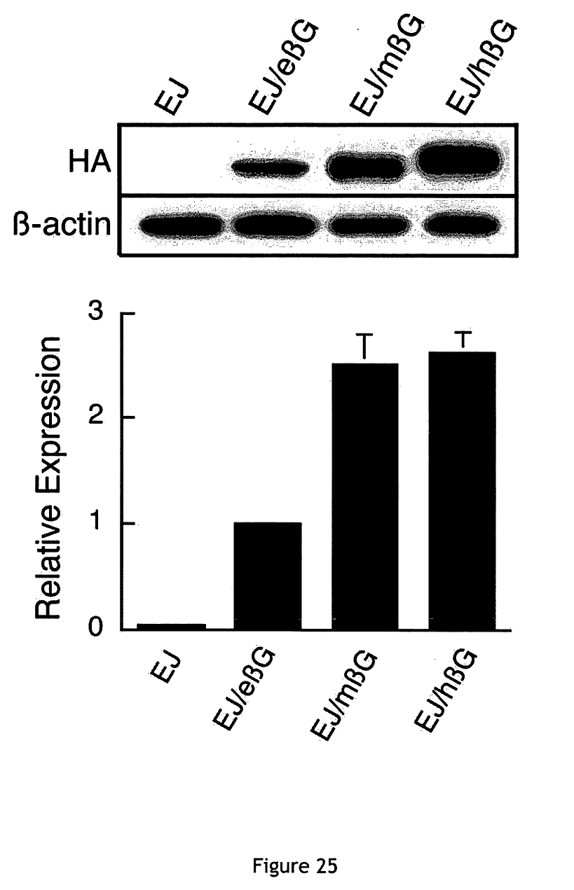

FIG. 25 shows the immunoblot results of characterization of exemplary membrane-bound β-glucuronidase expression in stable EJ bladder carcinoma cells. Retroviral transduction of EJ human bladder cancer cells generated stable EJ transfectants that expressed hβG-e-B7, mβG-e-B7 or eβG-e-B7 (EJ/hβG, EJ/mβG and EJ/eβG cells, respectively). These stable EJ transfectants were immunoblotted with an anti-HA or anti-β-actin antibody. The relative levels of β-glucuronidase expression were estimated by normalizing the HA band intensity with the intensity of the β-actin band. Immunoblotting of whole cell lysates demonstrated that hβG-e-B7 and mβG-e-B7 were expressed at about 2.5 fold higher levels as compared to eβG-e-B7.

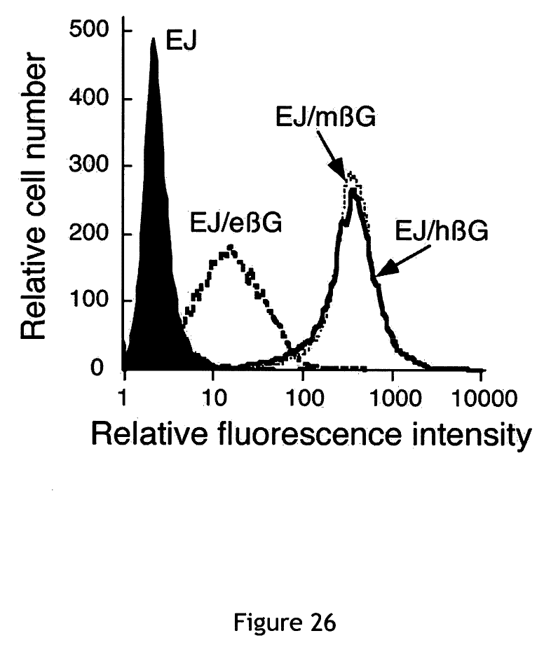

FIG. 26 shows the results of immunofluorescence as analyzed on a flow cytometer by expressing various exemplary hβG-e-B7, mβG-e-B7 or eβG-e-B7 membrane bound β-glucuronidase reporter systems in live EJ cells and immunofluorescence staining for the HA epitope. High expression levels for both the recombinant hβG-e-B7 and mβG-e-B7 membrane bound proteins were detected on the surface of live EJ transfected cells.

The expression levels of the recombinant eβG-e-B7 proteins on the plasma membranes were about 20 fold lower than the recombinant hβG-e-B7 and mβG-e-B7 proteins. Comparing the results of transfected murine fibroblasts and murine colon carcinoma CT26 cells, the surface expression levels of E. coli β-glucuronidase are consistently lower than the surface expression levels of human β-glucuronidase and mouse β-glucuronidase.

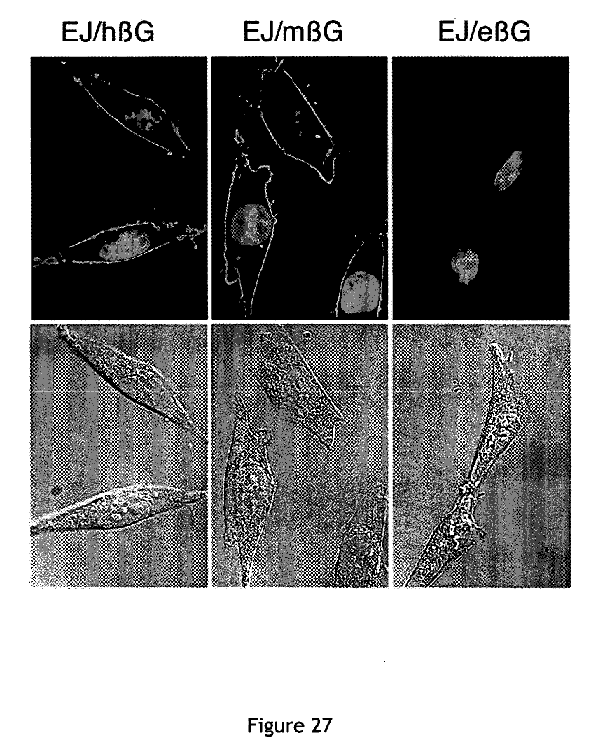

FIG. 27 shows the results of immunofluorescence as analyzed under a fluorescence microscope equipped with a CCD detector (upper panels) or under phase-contrast (lower panels). Blue regions show nuclear staining by DAPI. As shown in FIG. 27, by expressing various exemplary functional membrane bound β-glucuronidase reporter systems in live EJ cells, the expression of the recombinant hβG-e-B7 and mβG-e-B7 protein can be easily visualized on the plasma membrane of EJ cells whereas eβG-e-B7 is weakly detected.

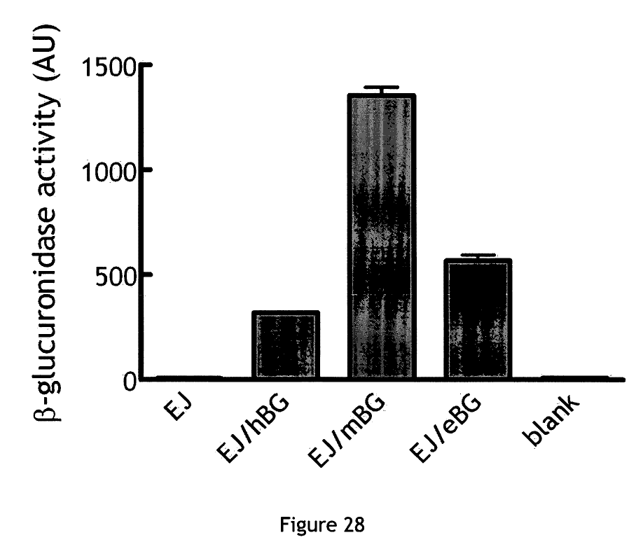

FIG. 28 shows the results of surface β-glucuronidase enzyme activity of exemplary βG-e-B7, mβG-e-B7 or eβG-e-B7 membrane-bound β-glucuronidase recombinant constructs in EJ cells. The results shown are the mean values of triplicate determinations. EJ/mβG cells display about 2-3 fold more β-glucuronidase activity than EJ/eβG cells and about 5-fold higher activity than EJ/hβG cells. As expected, EJ cells alone do not hydrolyze the glucuronide substrate. The enzymatic activity of E. coli β-glucuronidase in EJ cells are relatively higher than human β-glucuronidase, even though the expression levels of E. coli β-glucuronidase present on EJ/eβG cells are relatively lower than the expression levels of human β-glucuronidase.

### Example 10

### The Activities of Purified Human Mouse and E. coli β-Glucuronidase Recombinant Proteins

It is contemplated that the disparity between the relative surface expression levels the relative enzymatic activity of human β-glucuronidase, mouse β-glucuronidase, and E. coli β-glucuronidase in EJ cells is probably because of the neutral pH optimum of E. coli β-glucuronidase or a highly specific enzyme activity for the recombinant E. coli β-glucuronidase enzyme, which could cause the relatively effective hydrolysis of glucuronide substrates even though even though the expression levels of E. coli β-glucuronidase present on EJ/eβG cells are relatively lower than the expression levels of human β-glucuronidase. To differentiate these possibilities, recombinant forms of each enzyme are purified in order to measure accurately their specific activities at defined pH values.

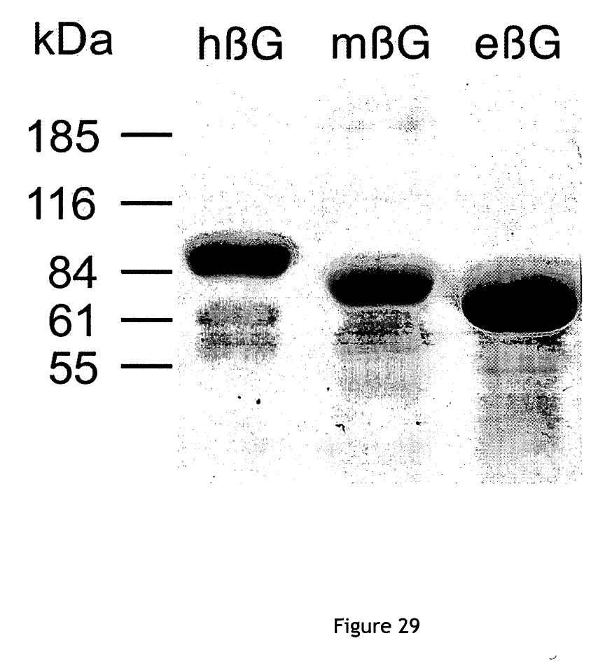

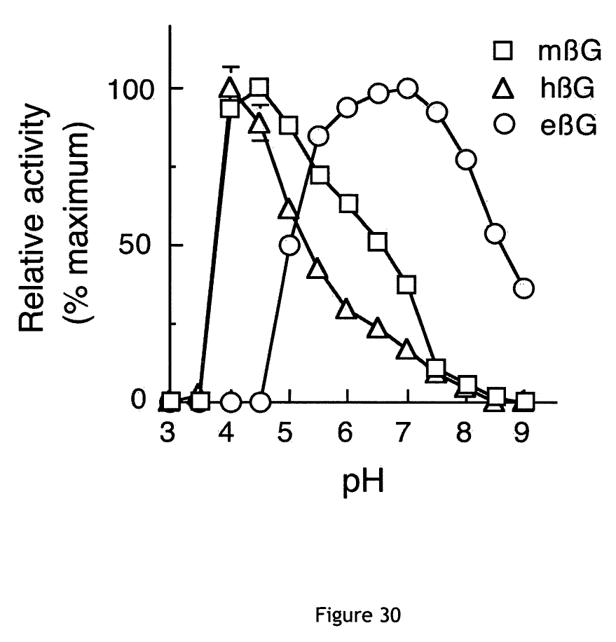

FIG. 29 shows an SDS-PAGE gel electrophoresis of the purified human, mouse, and E. coli β-glucuronidase recombinant proteins after staining the gel with Coomassie blue. FIG. 30 shows relative enzymatic activities for the purified human, mouse, and E. coli β-glucuronidase recombinant proteins at the indicated pH values (n=3). As expected, E. coli β-glucuronidase display optimal catalytic activity at neutral pH range, whereas both human β-glucuronidase and mouse β-glucuronidase exhibit maximal enzymatic activities at a pH range of about 4 to about 4.5. The relative enzymatic activities (percentage maximum activity) of these recombinant enzymes at the indicated pH values are shown (n=3).

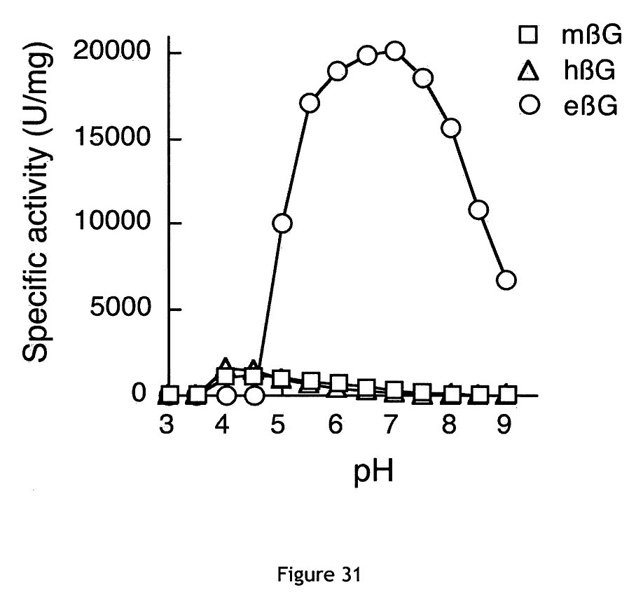

FIG. 31 shows the specific activity of the recombinant human, mouse, and E. coli β-glucuronidase recombinant proteins, at the indicated pH values (n=3). E. coli β-glucuronidase display a maximal specific activity of about 20,000 U/mg whereas human β-glucuronidase and mouse β-glucuronidase had maximal specific activities of abut 1600 U/mg and about 1100 U/mg, respectively. Thus, E. coli β-glucuronidase possesses intrinsically higher catalytic activity as compared to the lysosomal human β-glucuronidase and mouse β-glucuronidase enzymes.

### Example 11

### TM Domains of Type II Integral Membrane Proteins can be Used to Anchor β-Glucuronidase on the Cell Surface

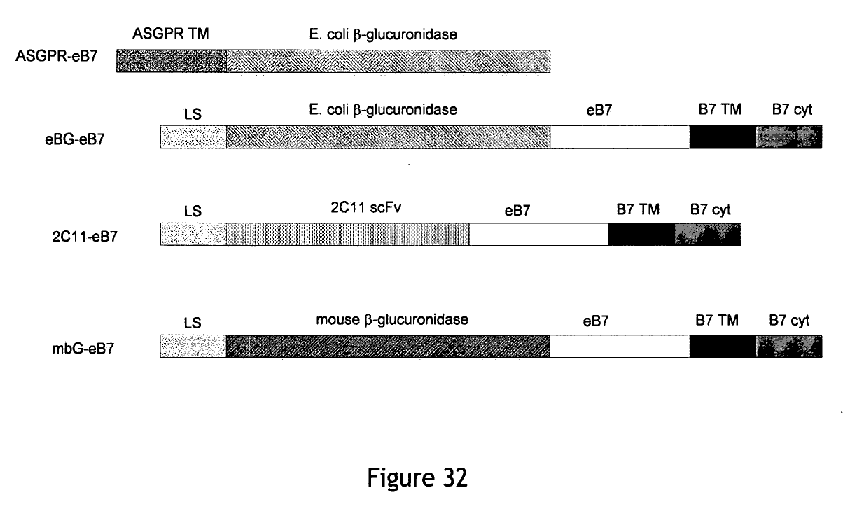

FIG. 32 illustrates linking of an exemplary transmembrane domain from a type II integral membrane protein, ASGPR, to E. coli β-glucuronidase and expressing the membrane-bound β-glucuronidase in 3T3 cells. A cDNA fragment encoding the type II transmembrane domain from the human asialoglycoprotein receptor (ASGPR) was fused in frame to the 5′ end of the E coli β-glucuronidase gene to create ASGPR-eβG. In addition, an immunoglobulin leader sequence was fused to the 5′ end of E. coli β-glucuronidase (eβG-e-B7), a single chain antibody (2C11-e-B7) or murine β-glucuronidase (mβG-e-B7), followed by a spacer domain containing the Ig-like C2-type and Ig-hinge-like domains, a transmembrane domain and a cytoplasmic tail of the murine B7-1 antigen. The type II transmembrane domain derived from the asialoglycoprotein receptor allowed E. coli β-glucuronidase to be expressed on 3T3 cells as shown by the high β-glucuronidase activity.

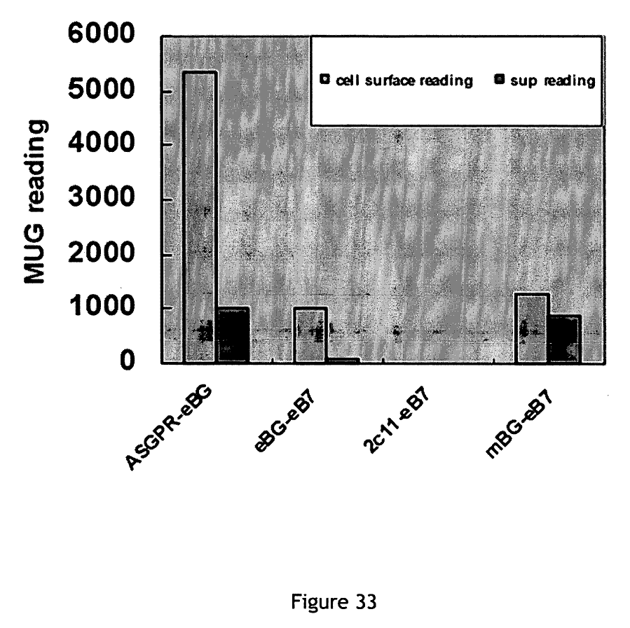

FIG. 33 shows the results of the glucuronidase activity for various exemplary recombinant constructs as shown in FIG. 32. 3T3 fibroblasts were transfected with plasmid DNAs using lipofectamine. After about 48 hour, the transfected cells were washed with phosphate-buffered saline and immediately assayed for β-glucuronidase activity by adding 4-methylumbelliferyl β-D-glucuronide for about 30 min at 37° C. The fluorescence (MUG reading as shown) of the resulting mixture was measured. The β-glucuronidase activity in the culture medium of transfected cells was also measured in an analogous way. High β-glucuronidase activity was detected on the surfaces of live 3T3 cells after E. coli β-glucuronidase was anchored to their surface.

### Example 12

### Linking of β-Glucuronidase to an GPI Anchor

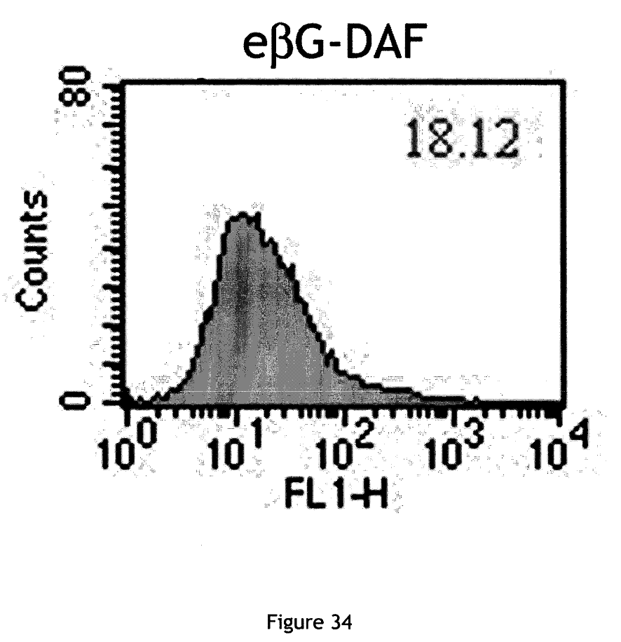

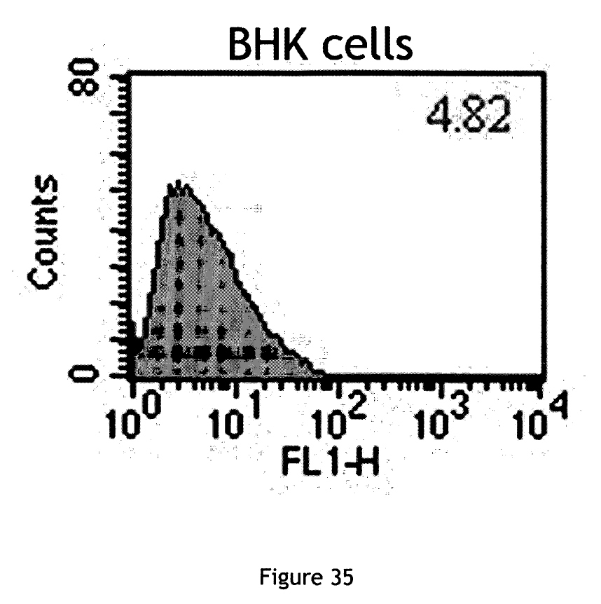

FIG. 34 shows immunofluorescence of live BHK cells expressing GPI-anchored E. coli β-glucuronidase as analyzed on a flow cytometer by staining for the presence of β-glucuronidase with an anti-β-glucuronidase antibody and FIG. 35 shows the control immunofluorescence of endogenous β-glucuronidase staining in live BHK cells as analyzed a flow cytometer. The exemplary GPI anchor used derived from human decay accelerating factor (DAF). The resulting reporter gene system was constructed as an expression plasmid containing an immunoglobulin leader sequence placed at the 5′ end of the E. coli β-glucuronidase gene followed by a DNA fragment encoding the last 37 amino acids of human decay accelerating factor (DAF). BHK cells were transiently transfected with the expression plasmid coding for a recombinant E. coli β-glucuronidase-DAF fusion protein. Two days later, untransfected BHK cells or BHK cells transfected with E. coli β-glucuronidase-DAF were immunofluorescence stained for β-glucuronidase expression.

### Example 13

### β-Glucuronidase and its Substrates

In mammals, glucuronidation is a principle means of detoxifying or inactivating compounds that utilizes UDP glucuronyl transferase systems. Many β-glucuronides can be prepared free of other contaminating glycosides by vigorous acid hydrolysis, which cleaves glucosides, galactosides and other glycosides, but leaves glucuronides mostly intact. β-glucuronides are extremely important as the principle form in which xenobiotics and endogenous phenols and aliphatic alcohols are excreted in the urine and bile of vertebrates. Colorigenic and fluorogenic glucorogenic substrates, such as p-nitrophenyl β-D-glucuronide and 4-methylumbelliferyl β-D-glucuronide are much more stable in aqueous solution than other glycosides such as β-D-galactosides or β-D-glucosides.

β-glucuronidase activity is reliably reported almost exclusively from those organisms that have, or are associated with organisms that have glucuronidation as a detoxification pathway. For example, all vertebrates use glucuronidation as the principle conjugation mechanism, together with some of their endogenous microbe populations (usually E. coli) have β-glucuronidase (GUS) activity.

β-glucuronidase catalyzes the hydrolysis of a very wide variety of β-glucuronides, and E. coli β-glucuronidase also hydrolyses with much lower efficiency some β-galacturonides. In general, β-glucuronidase is very stable and can tolerate many detergents under widely varying ionic conditions.

β-glucuronidase activity is extremely common in almost all tissues of all vertebrates and many mollusks. β-glucuronidase enzymes can be purified from many mammalian sources and demonstrate to be homotetramer in structure, with a subunit molecular weight of approximately 70 kDa. The enzymes from mammalian sources are synthesized with a signal sequence at the amino terminus, and then transported to and glycosylated within the endoplasmic reticulum and ultimately localized within vacuoles intracellularly. Unlike bacterial β-glucuronidase, mammalian and molluscan β-glucuronidase can cleave thioglucuronides. In general, however, the E. coli β-glucuronidase is much more active than the mammalian β-glucuronidase enzyme against most biosynthetically derived β-glucuronides. β-glucuronidase activity is largely if not completely absent from higher plants.

Suitable substrates for β-glucuronidase may include a glucuronide functional group in addition to a chromogenic, fluorescent, colorigenic, or radioactive group capable of being hydrolyzed into a chromogenic, fluorescent, colorigenic, or radioactive reporter product. In general, the substrates of β-glucuronidase used in the membrane bound reporter gene systems may be non-chromogenic, non-fluorescent, or non-colorigenic, and may be converted into chromogenic, fluorescent, or colorigenic products to be imaged or assayed conveniently. Synthetic substrates for β-glucuronidase can be generated to assist in vitro assaying and in vivo imaging of the expression of the recombinant reporter gene systems as described herein by assaying the activity of the β-glucuronidase enzyme. For example, a non-fluorescent substrate for β-glucuronidase, fluorescein di-β-D-glucuronide (FDGlcU), may be used in the membrane bound reporter gene system and can be converted to a fluorescent product, fluorescein, in vitro and in vivo.

### Example 14

### Synthetic β-Glucuronidase Substrates for In Vivo PET Imaging

Additional detection systems compatible with the membrane bound reporter gene systems can also be used such that the recombinant reporter gene constructs can also be detected in these systems by designing different substrates/probes. For example, positron emission tomography (PET) is a nuclear medical imaging technique which produces a three-dimensional image or map of functional processes in the body. A molecule containing a short-lived radioactive tracer isotope (TRAP), which decays by emitting a positron, is injected and allowed to accumulate in the target tissues. When an emitted positron encounters and annihilates an electron, a pair of photons traveling in opposite directions is created. The photons are then detected in a scanning device. Synthetic substrates for β-glucuronidase generated to assist in vivo imaging for the expression of the recombinant reporter gene system may include a glucuronide substrate compatible with a TRAP moiety to allow trapping of a radioactive side chain or a radioactive conjugate for measuring the photon emissions of the resulting radioactive TRAP product. Hydrolysis of the glucuronide group of the radioactive conjugate increases its reactivity with proteins so that it will rapidly be covalently bound to proteins near the activation site. In another embodiment, hydrolysis of the glucuronide moiety produces a water insoluble reaction product that can be locally retained by precipitating or dissolving into lipophilic structures such as the plasma membrane of cells. More descriptions of PET imaging systems can be found under http://en.wikipedia.org/wiki/Positron_emission_tomography.

Suitable TRAP compatible substrates for β-glucuronidase (βG) can be designed and used in the membrane bound reporter gene systems. For example, a radioactive TRAP conjugate, such as difluoromethylphenol-¹²⁴I (trap-I¹²⁴), can be conjugated with a functional glucuronide group to generate a substrate for β-glucuronidase, difluoromethylphenol-¹²⁴I-glucuronide (I¹²⁴-gluc). The radioactivity or photon emission of the radioactive side chain or radioactive conjugate, I¹²⁴, can be measured after β-glucuronidase activation, which releases the radioactive product, difluoromethylphenol-I¹²⁴, which is retained at the site of β-glucuronidase hydrolysis.

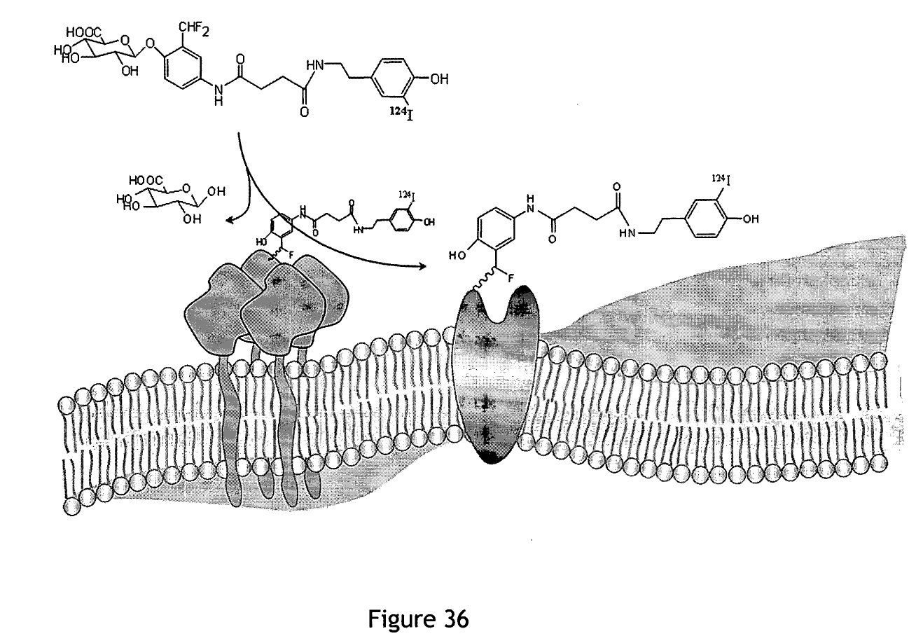

FIG. 36 shows in vivo imaging by targeted activation of a glucuronide TRAP compatible substrate/probe, difluoromethylphenol-¹²⁴I glucuronide (¹²⁴I-trap-glu), which can be enzymatically converted to an active trap-¹²⁴I by membrane bound β-glucuronidase to assess the location and persistence of gene expression in vivo.

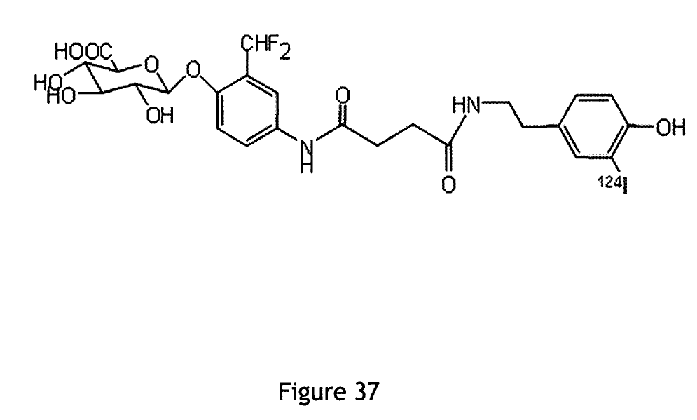

FIG. 37 illustrates the chemical structure of the ¹²⁴I-difluoromethylphenol glucuronide probe (¹²⁴I-trap-glu) according to one embodiment of the invention. Accordingly, recombinant membrane bound β-glucuronidase enzymes are capable of converting a TRAP-compatible substrate into a TRAP compatible reporter product. For example, the TRAP-compatible substrate may include, but not limited to, a radioactive TRAP compatible substrate, a fluorescent TRAP compatible substrate, FITC-trap-glu, ¹²⁴I-difluoromethylphenol glucuronide (¹²⁴I-trap-glu), ¹²⁴I-phenolphthalin glucuronide (¹²⁴I-ph-glu), and combinations thereof.

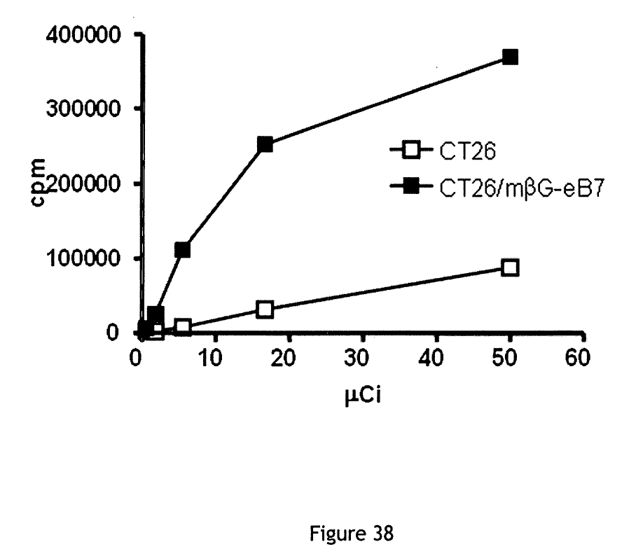

FIG. 38 shows the results demonstrating the specificity of mβG-eB7, which specifically converts the ¹²⁴I-difluoromethylphenol glucuronide probe (¹²⁴I-trap-glu) to ¹²⁴I-trap product in CT26/mβG-eB7 (▪) but not in CT26 cells (□). Graded concentrations of the ¹²⁴I-trap-glu probe/substrate were incubated with CT26/mβG-eB7 (▪) or CT26 cells (□) in phosphate-buffer saline at room temperature for about 40 mins. The plates containing the transfected cells were washed and bound radioactivity was measured in a gamma-counter.

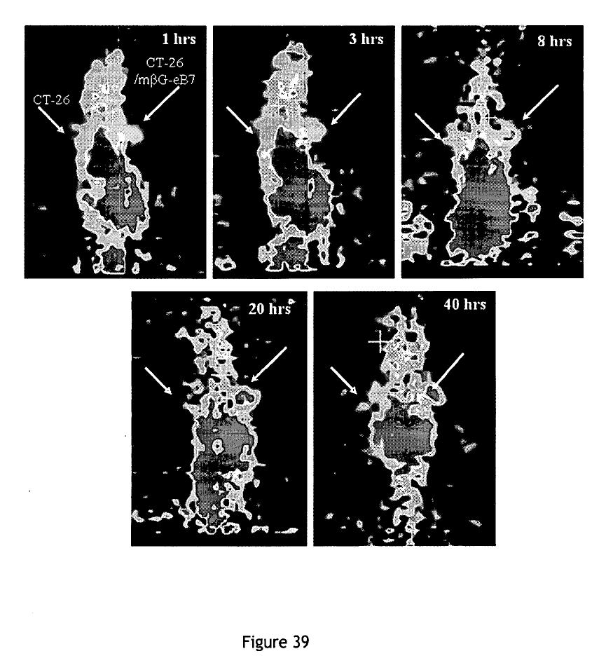

FIG. 39 shows the results of in vivo micro-PET imaging of membrane bound β-glucuronidase (βG) gene expression by ¹²⁴I-trap-glu. About 100 μCi of the ¹²⁴I-trap-glu substrate was i.v. injected into mice. Whole-body scintigraphy of pentobarbital-anesthetized mice was performed at about 1 hour, 3 hours, 8 hours, 20 hours, and 40 hours with a micro-PET (Concorde microPET R4) instrument. As another example, ¹²⁴I-phenolphthalin glucuronide (¹²⁴I-ph-glu) can be used as a substrate/probe for reporter systems expressing β-glucuronidase.

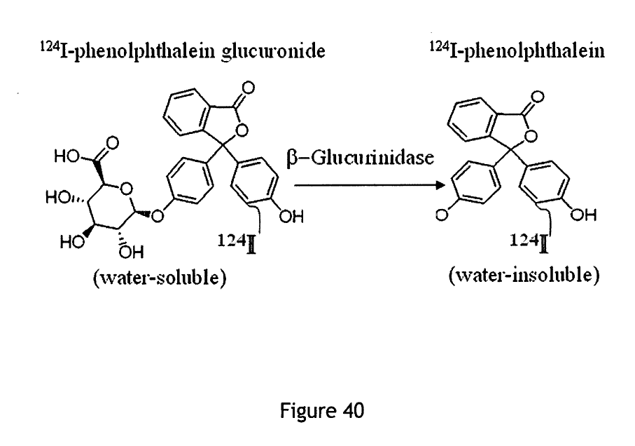

FIG. 40 illustrates the chemical structure of the ¹²⁴I-phenolphthalin glucuronide probe (¹²⁴I-ph-trap-glu). The ¹²⁴I-trap-glu probe can be enzymatically hydrolyzed by β-glucuronidase through the hydrophilic glucuronide group to produce a water-insoluble ¹²⁴I-phenolphthalin reporter product, useful for assessing the location and persistence of gene expression.

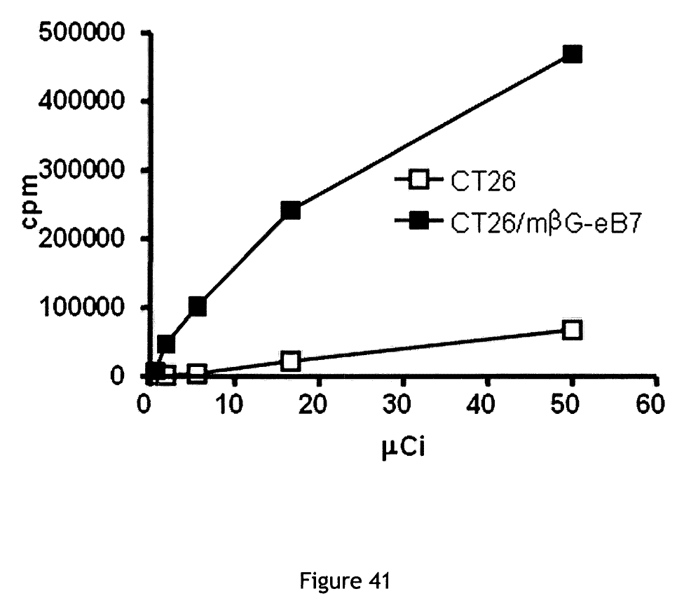

FIG. 41 shows the results demonstrating the specificity of the ¹²⁴I-trap-glu probe. Graded concentrations of the ¹²⁴I-trap-glu probe was added to 96-well microtiter plates coated with CT26/mβG-eB7 (▪) or CT26 cells (□) in phosphate-buffered saline at room temperature for about 40 mins. The plates were then washed to remove non-precipitated probes and the cells were collected by treatment with trypsin. The radioactivity of cells was then measured in a gamma-counter. As shown in FIG. 41, mβG-eB7 specifically converts the ¹²⁴I-phenolphthalin glucuronide probe (¹²⁴I-ph-trap-glu) into a ¹²⁴I-trap reporter product in CT26/mβG-eB7 cells (▪) but not in CT26 cells (□).

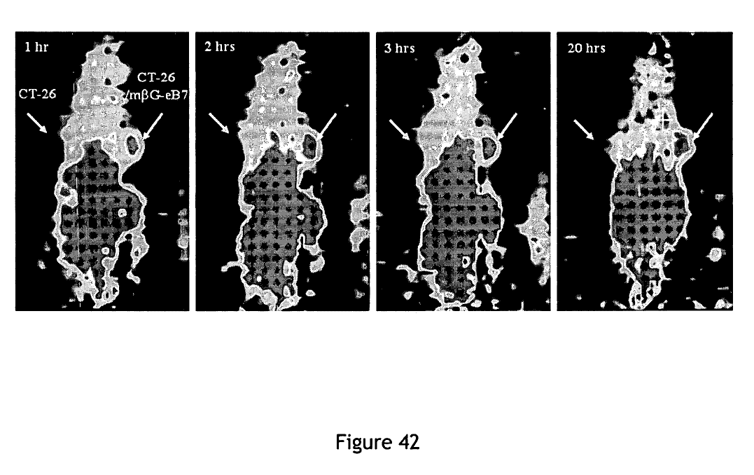

FIG. 42 shows the results of in vivo micro-PET imaging of membrane bound β-glucuronidase (βG) gene expression by ¹²⁴I-ph-trap-glu. About 100 μCi of ¹²⁴I-ph-glu was i.v. injected into mice. Whole-body scintigraphy of pentobarbital-anesthetized mice was performed at about 1 hour, 3 hours, 8 hours, and 20 hours with a micro-PET (Concorde microPET R4) instrument.

As another example, FITC-trap-glu (N-fluorescein-isothiocyanato (3-difluoromethylphenyl)-β-D-glucopyronuronate), which can be used in the membrane bound reporter gene system as the substrate for β-glucuronidase to be converted into FITC-trap (N-fluorescein-isothiocyanato (3-difluoromethylphenyl) for assessing the location and persistence of in vivo gene expression after β-glucuronidase activation. Hydrolysis of the glucuronide moiety in FITC-trap-Glu increases the reactivity of the difluoromethylphenyl group so that it can covalently react and attach to proteins near the site of hydrolysis. This allows retention of the FITC-trap at the sites where β-glucuronidase is expressed as a membrane protein on cells.

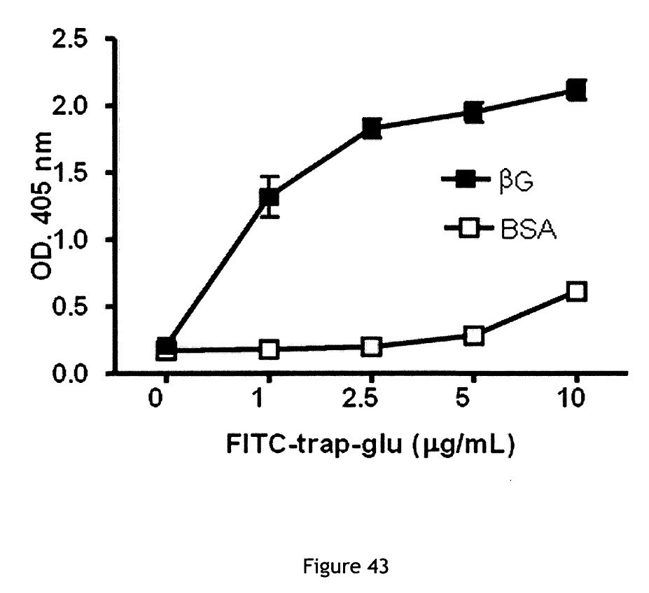

FIG. 43 demonstrates specific trapping of FITC-trap-glu by β-glucuronidase as detected by an anti-FITC antibody. Recombinant E. coli β-glucuronidase (about 2 μg/well) and bovine serum albumin (about 2 μg/well) were coated in 96 well microtiter plates and blocked with about 2% of skin milk. Serial dilutions of the FITC-trap-glu probe in phosphate-buffered saline were added to the wells of the microtiter plates at room temperature for about 60 mins. The plates were washed 3 times with phosphate-buffered saline. The plates were stained with about 1 μg/mL of an anti-FITC antibody followed by about 1 μg/mL of horse-radish peroxidase-conjugated anti-mouse IgG. The plates were washed and bound peroxidase activity was measured by adding about 100 μL/well of ABTS solution (about 0.4 mg/mL of 2,2′ azino-di(3-ethylbenzthiazoline-6-sulfonic acid), about 0.003% of H₂O₂, about 100 mM of phosphate-citrate, pH 4.0) for about 30 min at room temperature.

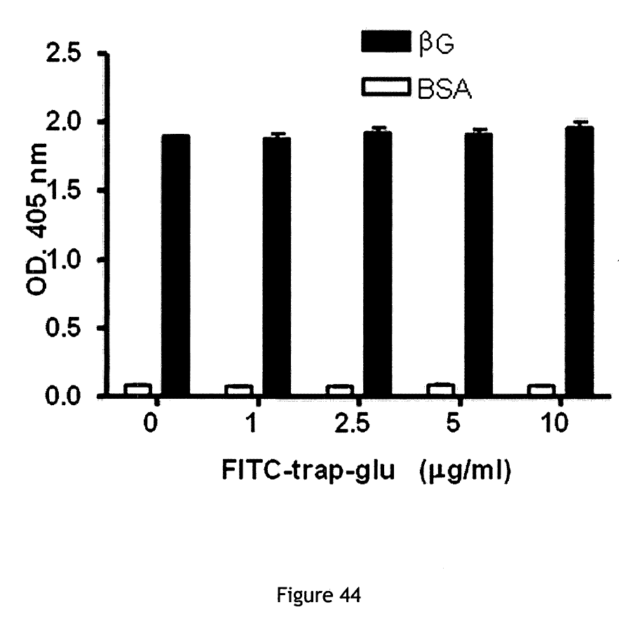

FIG. 44 shows the results of the measured β-glucuronidase activities at different concentrations of FITC-trap-glu. β-glucuronidase activity was measured in a microtiter plate by adding about 200 μl of phosphate-buffered saline (pH 7.5) containing about 0.1% of BSA and about 3.2 mM of p-nitrophenol β-D-glucuronide for about 30 min at 37° C. The absorbance value at 405 nm for each well was measured in a microplate reader (Molecular Device, Menlo Park, Calif.).

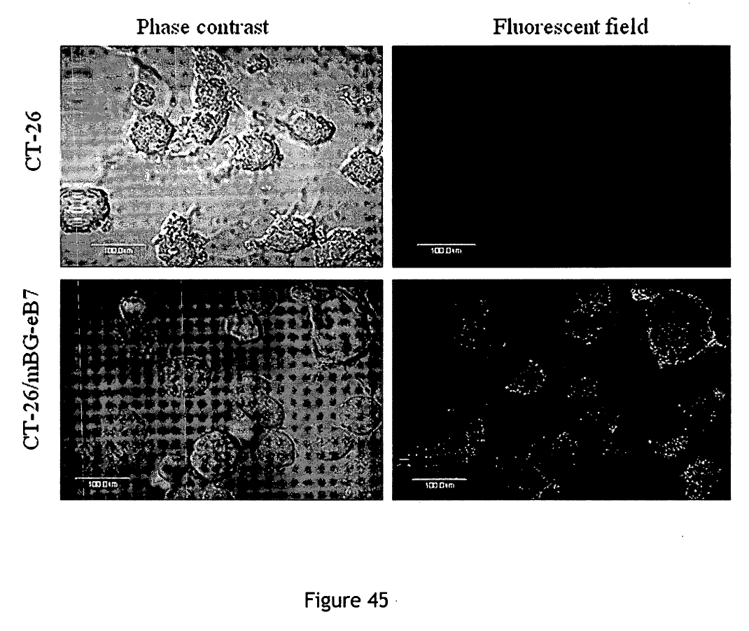

FIG. 45 demonstrates the specificity of FITC-trap-glu activation in β-glucuronidase-expressing cells in vitro as detected by an anti-FITC antibody and observed under phase contrast and fluorescent field confocal microscopes. Live CT26 and CT26/mβG-eB7 cells were incubated with about 0.5 μg/μl of the FITC-trap-glu probe in phosphate-buffered saline at room temperature for about 60 mins. After washing, the cells were stained with about 1 μg/mL of an anti-FITC antibody (sigma F5636) followed by about 1 μg/mL of a FITC-conjugated anti-mouse IgG. The cells were washed with cold phosphate-buffered saline, mounted with fluorescence mounting medium (DakoCytomation, Carpinteria, Calif.), and viewed under a digital fluorescence confocal microscope.

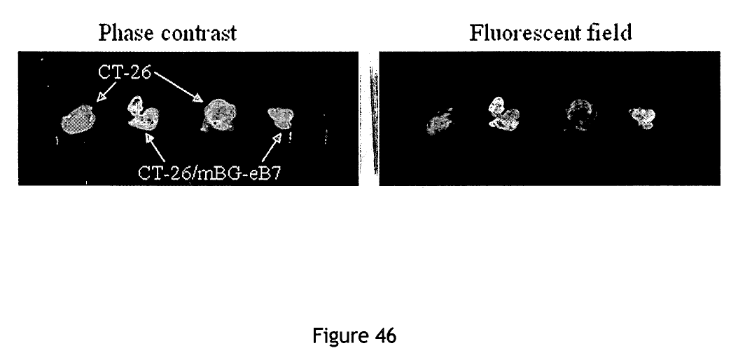

FIG. 46 shows the results of demonstrating the specificity of FITC-trap-glu activation in β-glucuronidase-expressing cells in vivo as detected by iv injection the substrate FITC-trap-glu and observed under phase contrast and fluorescent field confocal microscope. Balb/c mice bearing established CT26 and CT26/mβG-eB7 tumors (100-200 mm³) in their left and right chest regions, respectively, were i.v. injected with about 500 μg of FITC-trap-glu. The resulting tumors were excised at about 60 minutes after injection and imaged on a Kodak IS2000MM optical imaging system.

Addition suitable exemplary substrates include, but are not limited to, fluorescein di-β-D-glucuronide (FDGlcU), 9H-(1,3-dichloro-9,9-dimethylacridin-2-one-7-yl) β-D-glucuronide (DDAO GlcU), ELF® 97 β-D-glucuronidase substrate (ELF® 97 β-D-glucuronide), ImaGene Green™ C₁₂FDGlcU from GUS Gene Expression Kit, 4-methylumbelliferyl β-D-glucuronide (MUGlcU), 5-(pentafluorobenzoylamino)fluorescein di-β-D-glucopyranoside (PFB-FDGlu), 5-(pentafluorobenzoylamino)fluorescein di-β-D-glucuronide (PFB-FDGlcU), resorufin β-D-glucuronide, and β-trifluoromethylumbelliferyl β-D-glucuronide, etc.

The membrane bound reporter system as described herein using βG is based on several factors including: the low immunogenicity of endogenous βG to allow persistent imaging of gene expression; inaccessibility of glucuronides to endogenous lysosomal βG and low serum concentrations of βG, resulting in little non-specific probe activation; low toxicity of glucuronide conjugates due to their poor transport across the lipid bilayers of cells; rapid clearance from the blood allowing quicker imaging; signal amplification due to the catalytic hydrolysis of probe molecules; possibility of generating a range of imaging probes by attachment of glucuronide groups; and ability to perform imaging and gene therapy using the same recombinant DNA construct, among others. Based on these advantages, the βG imaging system appears to possess great potential for monitoring gene expression in animals and humans.

While the foregoing is directed to embodiments of the present invention, other and further embodiments of the invention may be devised without departing from the basic scope thereof, and the scope thereof is determined by the claims that follow.

1. A recombinant DNA molecule comprising a first DNA fragment encoding a β-glucuronidase and a second DNA fragment encoding a membrane anchoring domain.

2. The recombinant DNA of claim 1, wherein the membrane anchoring domain comprises an anchor selected from the group consisting of GPI (glycosylphosphatidylinositol) anchor, decay accelerating factor, CDw52, CD55, CD59, thy-1, and combinations thereof.

3. The recombinant DNA of claim 1, wherein the membrane anchoring domain comprises a transmembrane domain of an integral membrane protein, and wherein the integral membrane protein is selected from the group consisting of type I integral membrane proteins, type II integral membrane proteins, type III integral membrane proteins, membrane bound receptor proteins, a murine B7-1 antigen (e-B7), platelet-derived growth factor receptor (PDGFR), intracellular adhesion molecule 1 (ICAM-1), asialoglycoprotein receptor (ASGPR), aminopeptidase N (CD13), mast-cell function-associated antigen, influenza virus neuraminidase, dipeptidyl aminopeptidase IV (CD26), and combinations thereof.

4. The recombinant DNA of claim 1, further comprising a DNA fragment of a spacer domain.

5. The recombinant DNA of claim 4, wherein the spacer domain is selected from the group consisting of a myc epitope, a HA epitope, a flag epitope, flexible polypeptides, an extracellular domain of a membrane protein, an extracellular domain of murine B7-1 protein, Ig-like C2-type and Ig-hinge-like domains (e) of CD80 (B7-1 protein), Ig-like C2-type and Ig-hinge-like domains of murine B7-1 antigen (e-B7), hinge-CH₂-CH₃ domain of human IgG1 protein, hinge CH₂-CH₃ domains of an immunoglobulin protein (IgG1), CH₂-CH₃ domains of IgG1 (mγ₁), CH₂-CH₃ domains (lacking the hinge domain) of a human IgG1, first immunoglobulin-like V-type domain of human biliary glycoprotein I (BGP), N-terminal Ig-like V-type domain of biliary glycoprotein-1 (BGP-1), a BGP-1 extracellular protein domain, an extracellular portion of human CD44E, and combinations thereof.

6. The recombinant DNA of claim 5, wherein the spacer domain further comprises one or more O-linked or N-linked glycosylation sites.

7. The recombinant DNA of claim 1, further comprising a DNA fragment of a cytosolic domain of a membrane protein.

8. The recombinant DNA of claim 1, further comprising a DNA fragment of a leader sequence of a protein.

9. The recombinant DNA of claim 1, further comprising a DNA fragment of a synthetic linker domain.

10. The recombinant DNA of claim 1, wherein the β-glucuronidase is selected from the group consisting of human β-glucuronidase, mouse β-glucuronidase, and E. coli β-glucuronidase, and combinations thereof.

11. The recombinant DNA of claim 1, further comprising a DNA fragment encoding a product of an exogenous gene of interest.

12. The recombinant DNA of claim 1, further comprising a regulatory DNA region for the expression of an exogenous gene of interest.

13. The recombinant DNA of claim 1, wherein the β-glucuronidase is capable of converting a non-fluorescent substrate into a fluorescent report product.

14. The recombinant DNA of claim 1, wherein the β-glucuronidase is capable of converting a TRAP compatible substrate into a TRAP compatible report product.

15. The recombinant DNA of claim 14, wherein the TRAP-compatible substrate is selected from the group consisting of a radioactive TRAP compatible substrate, a fluorescent TRAP compatible substrate, FITC-trap-glu, ¹²⁴I-difluoromethylphenol glucuronide (¹²⁴I-trap-glu), ¹²⁴I-phenolphthalin glucuronide (¹²⁴I-ph-glu), and combinations thereof.

16. A method of introducing a gene of interest or portion thereof into a host cell, comprising:
introducing into the host cell a recombinant DNA construct, the recombinant DNA construct comprising
a first DNA fragment encoding a β-glucuronidase; and
a second DNA fragment encoding a membrane anchoring domain.

17. The method of claim 16, wherein the recombinant DNA construct further comprises a DNA sequence for the gene of interest or portions thereof;

18. The method of claim 16, wherein the recombinant DNA construct further comprises a DNA fragment of a leader sequence of a protein.

19. The method of claim 16, wherein the DNA sequence for the gene of interest comprises a regulatory DNA region for the expression of the gene of interest.

20. The method of claim 16, wherein the membrane anchoring domain comprises an anchor selected from the group consisting of GPI (glycosylphosphatidylinositol) anchor, decay accelerating factor, CDw52, CD55, CD59, thy-1, and combinations thereof.

21. The method of claim 16, wherein the membrane anchoring domain comprises an transmembrane domain of an integral membrane protein, and wherein the integral membrane protein is selected from the group consisting of type I integral membrane proteins, type II integral membrane proteins, type III integral membrane proteins, membrane bound receptor proteins, a murine B7-1 antigen (e-B7), platelet-derived growth factor receptor (PDGFR), intracellular adhesion molecule 1 (ICAM-1), asialoglycoprotein receptor (ASGPR), aminopeptidase N (CD13), mast-cell function-associated antigen, influenza virus neuraminidase, dipeptidyl aminopeptidase IV (CD26), and combinations thereof.

22. The method of claim 16, wherein the β-glucuronidase is selected from the group consisting of human β-glucuronidase, mouse β-glucuronidase, and E. coli β-glucuronidase, and combinations thereof.

23. The method of claim 16, wherein the β-glucuronidase is capable of converting a non-fluorescent substrate to a fluorescent report product.

24. The method of claim 23, wherein the non-fluorescent substrate of β-glucuronidase is selected from the group consisting of fluorescein di-β-D-glucuronide (FDGlcU), 9H-(1,3-dichloro-9,9-dimethylacridin-2-one-7-yl) β-D-glucuronide (DDAO GlcU), ELF® 97 β-D-glucuronidase substrate (ELF® 97 β-D-glucuronide), ImaGene Green™ C₁₂FDGlcU from GUS Gene Expression Kit, 4-methylumbelliferyl β-D-glucuronide (MUGlcU), 5-(pentafluorobenzoylamino)fluorescein di-β-D-glucopyranoside (PFB-FDGlu), 5-(pentafluorobenzoylamino)fluorescein di-β-D-glucuronide (PFB-FDGlcU), resorufin β-D-glucuronide, β-trifluoromethylumbelliferyl β-D-glucuronide, and combinations thereof.

25. The method of claim 16, wherein the β-glucuronidase is capable of converting a TRAP-compatible substrate into a TRAP compatible report product.

26. The method of claim 25, wherein the TRAP-compatible substrate is selected from the group consisting of a radioactive TRAP compatible substrate, a fluorescent TRAP compatible substrate, FITC-trap-glu, ¹²⁴I-difluoromethylphenol glucuronide (¹²⁴I-trap-glu), ¹²⁴I-phenolphthalin glucuronide (¹²⁴I-ph-glu), and combinations thereof.

27. An expression vector for delivering a gene of interest or portion thereof into a host cell, comprising:
a first DNA fragment encoding a β-glucuronidase; and
a second DNA fragment encoding a membrane anchoring domain.

28. The expression vector of claim 27, further comprising a DNA sequence for the gene of interest.

29. A method of imaging the expression of a gene of interest in a host cell, comprising:
introducing into the host cell a recombinant DNA construct, the recombinant DNA construct comprising
a DNA sequence for the gene of interest or portions thereof;
a first DNA fragment encoding a β-glucuronidase; and
a second DNA fragment encoding a membrane anchoring domain;
providing a β-glucuronidase substrate capable of being converted into a report reporter product by the β-glucuronidase; and
monitoring the levels of the reporter product in the host cell.

30. The method of claim 29, wherein the recombinant DNA construct further comprises a regulatory DNA region for the expression of the gene of interest.

31. The method of claim 29, wherein the β-glucuronidase substrate is selected from the group consisting of fluorescein di-β-D-glucuronide (FDGlcU), 9H-(1,3-dichloro-9,9-dimethylacridin-2-one-7-yl) β-D-glucuronide (DDAO GlcU), ELF® 97 β-D-glucuronidase substrate (ELF® 97 β-D-glucuronide), ImaGene Green™ C₁₂FDGlcU from GUS Gene Expression Kit, 4-methylumbelliferyl β-D-glucuronide (MUGlcU), 5-(pentafluorobenzoylamino)fluorescein di-β-D-glucopyranoside (PFB-FDGlu), 5-(pentafluorobenzoylamino)fluorescein di-β-D-glucuronide (PFB-FDGlcU), resorufin β-D-glucuronide, β-trifluoromethylumbelliferyl β-D-glucuronide, and combinations thereof.

32. The method of claim 29, wherein the β-glucuronidase substrate is a TRAP-compatible substrate to be converted into a TRAP compatible report product, wherein the TRAP-compatible substrate is selected from the group consisting of a radioactive TRAP compatible substrate, a fluorescent TRAP compatible substrate, FITC-trap-glu, ¹²⁴I-difluoromethylphenol glucuronide (¹²⁴I-trap-glu), ¹²⁴I-phenolphthalin glucuronide (¹²⁴I-ph-glu), and combinations thereof.


SEQUENCE LISTING (10 sequences)

SEQ ID NO: 1 - DNA, 1887 bp, Homo sapiens
ctgcagggcgggatgctgtacccccaggagagcccgtcgcgggagtgcaaggagctggacggcctctggagcttccgcgccgacttctctgacaaccgacgccggggcttcgaggagcagtggtaccggcggccgctgtgggagtcaggccccaccgtggacatgccagttccctccagcttcaatgacatcagccaggactggcgtctgcggcattttgtcggctgggtgtggtacgaacgggaggtgatcctgccggagcgatggacccaggacctgcgcacaagagtggtgctgaggattggcagtgcccattcctatgccatcgtgtgggtgaatggggtcgacacgctagagcatgaggggggctacctccccttcgaggccgacatcagcaacctggtccaggtggggcccctgccctcccggctccgaatcactatcgccatcaacaacacactcacccccaccaccctgccaccagggaccatccaatacctgactgacacctccaagtatcccaagggttactttgtccagaacacatattttgactttttcaactacgctggactgcagcggtctgtacttctgtacacgacacccaccacctacatcgatgacatcaccgtcaccaccagcgtggagcaagacagtgggctggtgaattaccagatctctgtcaagggcagtaacctgttcaagttggaagtgcgtcttttggatgcagaaaacaaagtcgtggcgaatgggactgggacccagggccaacttaaggtgccaggtgtcagcctctggtggccgtacctgatgcacgaacgccctgcctatctgtattcattggaggtgcagctgactgcacagacgtcactggggcctgtgtctgacttctacacactccctgtggggatccgcactgtggctgtcaccaagagccagttcctcatcaatgggaaacctttctatttccacggtgtcaacaagcatgaggatgcggacatccgagggaagggcttcgactggccgctgctggtgaaggacttcaacctgcttcgctggcttggtgccaacgctttccgtaccagccactacccctatgcagaggaagtgatgcagatgtgtgaccgctatgggattgtggtcatcgatgagtgtcccggcgtgggcctggcgctgccgcagttcttcaacaacgtttctctgcatcaccacatgcaggtgatggaagaagtggtgcgtagggacaagaaccaccccgcggtcgtgatgtggtctgtggccaacgagcctgcgtcccacctagaatctgctggctactacttgaagatggtgatcgctcacaccaaatccttggacccctcccggcctgtgacctttgtgagcaactctaactatgcagcagacaagggggctccgtatgtggatgtgatctgtttgaacagctactactcttggtatcacgactacgggcacctggagttgattcagctgcagctggccacccagtttgagaactggtataagaagtatcagaagcccattattcagagcgagtatggagcagaaacgattgcagggtttcaccaggatccacctctgatgttcactgaagagtaccagaaaagtctgctagagcagtaccatctgggtctggatcaaaaacgcagaaaatatgtggttggagagctcatttggaattttgccgatttcatgactgaacagtcaccgacgagagtgctggggaataaaaaggggatcttcactcggcagagacaaccaaaaagtgcagcgttccttttgcgagagagatactggaagattgccaatgaaaccaggtatccccactcagtagccaagtcacaatgtttggaaaacagcccgtttact

SEQ ID NO: 2 - protein, 629 aa, Homo sapiens
LQGGMLYPQESPSRECKELDGLWSFRADFSDNRRRGFEEQWYRRPLWESGPTVDMPVPSSFNDISQDWRLRHFVGWVWYEREVILPERWTQDLRTRVVLRIGSAHSYAIVWVNGVDTLEHEGGYLPFEADISNLVQVGPLPSRLRITIAINNTLTPTTLPPGTIQYLTDTSKYPKGYFVQNTYFDFFNYAGLQRSVLLYTTPTTYIDDITVTTSVEQDSGLVNYQISVKGSNLFKLEVRLLDAENKVVANGTGTQGQLKVPGVSLWWPYLMHERPAYLYSLEVQLTAQTSLGPVSDFYTLPVGIRTVAVTKSQFLINGKPFYFHGVNKHEDADIRGKGFDWPLLVKDFNLLRWLGANAFRTSHYPYAEEVMQMCDRYGIVVIDECPGVGLALPQFFNNVSLHHHMQVMEEVVRRDKNHPAVVMWSVANEPASHLESAGYYLKMVIAHTKSLDPSRPVTFVSNSNYAADKGAPYVDVICLNSYYSWYHDYGHLELIQLQLATQFENWYKKYQKPIIQSEYGAETIAGFHQDPPLMFTEEYQKSLLEQYHLGLDQKRRKYVVGELIWNFADFMTEQSPTRVLGNKKGIFTRQRQPKSAAFLLRERYWKIANETRYPHSVAKSQCLENSPFT

SEQ ID NO: 3 - DNA, 1878 bp, Mus musculus
ctgaagggcgggatgctgttcccgaaggagagcccgtcgcgggagctcaaggcgctggacggactgtggcacttccgcgccgacctctcgaacaaccggctgcagggtttcgagcagcaatggtaccggcagccgctacgggagtcgggcccagtcttggacatgcctgtcccttctagcttcaatgacatcacccaagaagcagcccttcgggactttattggctgggtgtggtatgaacgggaagcaatcctgccacggcgatggacccaagataccgacatgagagtggtgttgaggatcaacagtgcccattattatgcagttgtgtgggtgaatgggattcatgtggtggaacatgagggaggtcacctcccctttgaggctgacattagcaagctggtccagagtgggcccctgaccacctgccggatcacgattgccatcaacaacacactgacccctcatacccttccgccggggaccatcgtctacaagactgacacctccatgtatcccaagggttactttgtccaggacacaagctttgacttcttcaactatgcgggactgcatcgatctgtggtcctctataccacccctaccacttacatcgatgatatcactgtgatcactaatgtggagcaagacatcgggctggtgacctactggatttctgtgcagggcagcgaacatttccagctagaggtgcaacttttggatgaggacggcaaagtcgtggcccatgggacagggaaccagggtcaacttcaggttcccagtgccaacctctggtggccttacctgatgcatgagcatccagcctacatgtactccttggaggtgaaggtgacaacaactgagtctgtgactgactactacacccttcctgtcgggattcgaacagtggctgtcacaaagagcaagttcctcataaacgggaagcccttctatttccaaggggtcaacaagcacgaggattcagatatccgagggaaaggcttcgactggccgctactggtaaaggatttcaacctgctccgttggctcggggcaaattcctttcgtaccagccactatccctactcagaggaggtacttcagctctgtgaccgatacgggattgtggtcatcgatgagtgtcccggtgtgggcattgtgctacctcagagttttggcaacgagtcacttcggcaccacctagaggtgatggaggagctggttcgccgggacaaaaatcaccctgcggttgtgatgtggtctgtggccaatgagccttcctctgctctgaaacccgccgcatattactttaagacgctgatcacccacaccaaagccctggacctcacccgtcccgtgacctttgtgagcaacgccaaatatgatgcagacctgggggccccgtacgtggatgttatctgtgtaaacagctacttttcttggtatcatgactatgggcatttggaggtgattcagccacagctgaatagccagtttgagaactggtataagacgcatcagaagccgattatccagagcgagtatggagcagacgcaatcccagggatccacgaggacccgcctcgcatgttcagtgaggagtaccagaaggctgttctggagaattaccattcagttctggatcagaaacgtaaagaatacgtggtcggagagctcatctggaatttcgccgacttcatgacgaaccagtcaccactgagagtaattggaaacaagaaggggatcttcactcgccagagacagcccaaaacttcggcctttattttgcgagagagatactggaggattgccaacgaaaccggaggtcacggttcagggccgaggacccagtgtttcggaagcagaccgttcacgttc

SEQ ID NO: 4 - protein, 626 aa, Mus musculus
LKGGMLFPKESPSRELKALDGLWHFRADLSNNRLQGFEQQWYRQPLRESGPVLDMPVPSSFNDITQEAALRDFIGWVWYEREAILPRRWTQDTDMRVVLRINSAHYYAVVWVNGIHVVEHEGGHLPFEADISKLVQSGPLTTCRITIAINNTLTPHTLPPGTIVYKTDTSMYPKGYFVQDTSFDFFNYAGLHRSVVLYTTPTTYIDDITVITNVEQDIGLVTYWISVQGSEHFQLEVQLLDEDGKVVAHGTGNQGQLQVPSANLWWPYLMHEHPAYMYSLEVKVTTTESVTDYYTLPVGIRTVAVTKSKFLINGKPFYFQGVNKHEDSDIRGKGFDWPLLVKDFNLLRWLGANSFRTSHYPYSEEVLQLCDRYGIVVIDECPGVGIVLPQSFGNESLRHHLEVMEELVRRDKNHPAVVMWSVANEPSSALKPAAYYFKTLITHTKALDLTRPVTFVSNAKYDADLGAPYVDVICVNSYFSWYHDYGHLEVIQPQLNSQFENWYKTHQKPIIQSEYGADAIPGIHEDPPRMFSEEYQKAVLENYHSVLDQKRKEYVVGELIWNFADFMTNQSPLRVIGNKKGIFTRQRQPKTSAFILRERYWRIANETGGHGSGPRTQCFGSRPFTF

SEQ ID NO: 5 - DNA, 36 bp, Artificial
  artificial myc epitope
gtcgacgaacaaaaactcatctcagaagaggatctg

SEQ ID NO: 6 - protein, 12 aa, Artificial
  artificial myc epitope translated
VDEQKLISEEDL

SEQ ID NO: 7 - DNA, 492 bp, Mus musculus
gctgacttctctacccccaacataactgagtctggaaacccatctgcagacactaaaaggattacctgctttgcttccgggggtttcccaaagcctcgcttctcttggttggaaaatggaagagaattacctggcatcaatacgacaatttcccaggatcctgaatctgaattgtacaccattagtagccaactagatttcaatacgactcgcaaccacaccattaagtgtctcattaaatatggagatgctcacgtgtcagaggacttcacctgggaaaaacccccagaagaccctcctgatagcaagaacacacttgtgctctttggggcaggattcggcgcagtaataacagtcgtcgtcatcgttgtcatcatcaaatgcttctgtaagcacagaagctgtttcagaagaaatgaggcaagcagagaaacaaacaacagccttaccttcgggcctgaagaagcattagctgaacagaccgtcttcctt

SEQ ID NO: 8 - protein, 164 aa, Mus musculus
ADFSTPNITESGNPSADTKRITCFASGGFPKPRFSWLENGRELPGINTTISQDPESELYTISSQLDFNTTRNHTIKCLIKYGDAHVSEDFTWEKPPEDPPDSKNTLVLFGAGFGAVITVVVIVVIIKCFCKHRSCFRRNEASRETNNSLTFGPEEALAEQTVFL

SEQ ID NO: 9 - DNA, 2406 bp, Artificial
  Artificial recombinant DNA
ctgaagggcgggatgctgttcccgaaggagagcccgtcgcgggagctcaaggcgctggacggactgtggcacttccgcgccgacctctcgaacaaccggctgcagggtttcgagcagcaatggtaccggcagccgctacgggagtcgggcccagtcttggacatgcctgtcccttctagcttcaatgacatcacccaagaagcagcccttcgggactttattggctgggtgtggtatgaacgggaagcaatcctgccacggcgatggacccaagataccgacatgagagtggtgttgaggatcaacagtgcccattattatgcagttgtgtgggtgaatgggattcatgtggtggaacatgagggaggtcacctcccctttgaggctgacattagcaagctggtccagagtgggcccctgaccacctgccggatcacgattgccatcaacaacacactgacccctcatacccttccgccggggaccatcgtctacaagactgacacctccatgtatcccaagggttactttgtccaggacacaagctttgacttcttcaactatgcgggactgcatcgatctgtggtcctctataccacccctaccacttacatcgatgatatcactgtgatcactaatgtggagcaagacatcgggctggtgacctactggatttctgtgcagggcagcgaacatttccagctagaggtgcaacttttggatgaggacggcaaagtcgtggcccatgggacagggaaccagggtcaacttcaggttcccagtgccaacctctggtggccttacctgatgcatgagcatccagcctacatgtactccttggaggtgaaggtgacaacaactgagtctgtgactgactactacacccttcctgtcgggattcgaacagtggctgtcacaaagagcaagttcctcataaacgggaagcccttctatttccaaggggtcaacaagcacgaggattcagatatccgagggaaaggcttcgactggccgctactggtaaaggatttcaacctgctccgttggctcggggcaaattcctttcgtaccagccactatccctactcagaggaggtacttcagctctgtgaccgatacgggattgtggtcatcgatgagtgtcccggtgtgggcattgtgctacctcagagttttggcaacgagtcacttcggcaccacctagaggtgatggaggagctggttcgccgggacaaaaatcaccctgcggttgtgatgtggtctgtggccaatgagccttcctctgctctgaaacccgccgcatattactttaagacgctgatcacccacaccaaagccctggacctcacccgtcccgtgacctttgtgagcaacgccaaatatgatgcagacctgggggccccgtacgtggatgttatctgtgtaaacagctacttttcttggtatcatgactatgggcatttggaggtgattcagccacagctgaatagccagtttgagaactggtataagacgcatcagaagccgattatccagagcgagtatggagcagacgcaatcccagggatccacgaggacccgcctcgcatgttcagtgaggagtaccagaaggctgttctggagaattaccattcagttctggatcagaaacgtaaagaatacgtggtcggagagctcatctggaatttcgccgacttcatgacgaaccagtcaccactgagagtaattggaaacaagaaggggatcttcactcgccagagacagcccaaaacttcggcctttattttgcgagagagatactggaggattgccaacgaaaccggaggtcacggttcagggccgaggacccagtgtttcggaagcagaccgttcacgttcgtcgacgaacaaaaactcatctcagaagaggatctggctgacttctctacccccaacataactgagtctggaaacccatctgcagacactaaaaggattacctgctttgcttccgggggtttcccaaagcctcgcttctcttggttggaaaatggaagagaattacctggcatcaatacgacaatttcccaggatcctgaatctgaattgtacaccattagtagccaactagatttcaatacgactcgcaaccacaccattaagtgtctcattaaatatggagatgctcacgtgtcagaggacttcacctgggaaaaacccccagaagaccctcctgatagcaagaacacacttgtgctctttggggcaggattcggcgcagtaataacagtcgtcgtcatcgttgtcatcatcaaatgcttctgtaagcacagaagctgtttcagaagaaatgaggcaagcagagaaacaaacaacagccttaccttcgggcctgaagaagcattagctgaacagaccgtcttcctt

SEQ ID NO: 10 - protein, 802 aa, Artificial
  artificial recombinant protein
LKGGMLFPKESPSRELKALDGLWHFRADLSNNRLQGFEQQWYRQPLRESGPVLDMPVPSSFNDITQEAALRDFIGWVWYEREAILPRRWTQDTDMRVVLRINSAHYYAVVWVNGIHVVEHEGGHLPFEADISKLVQSGPLTTCRITIAINNTLTPHTLPPGTIVYKTDTSMYPKGYFVQDTSFDFFNYAGLHRSVVLYTTPTTYIDDITVITNVEQDIGLVTYWISVQGSEHFQLEVQLLDEDGKVVAHGTGNQGQLQVPSANLWWPYLMHEHPAYMYSLEVKVTTTESVTDYYTLPVGIRTVAVTKSKFLINGKPFYFQGVNKHEDSDIRGKGFDWPLLVKDFNLLRWLGANSFRTSHYPYSEEVLQLCDRYGIVVIDECPGVGIVLPQSFGNESLRHHLEVMEELVRRDKNHPAVVMWSVANEPSSALKPAAYYFKTLITHTKALDLTRPVTFVSNAKYDADLGAPYVDVICVNSYFSWYHDYGHLEVIQPQLNSQFENWYKTHQKPIIQSEYGADAIPGIHEDPPRMFSEEYQKAVLENYHSVLDQKRKEYVVGELIWNFADFMTNQSPLRVIGNKKGIFTRQRQPKTSAFILRERYWRIANETGGHGSGPRTQCFGSRPFTFVDEQKLISEEDLADFSTPNITESGNPSADTKRITCFASGGFPKPRFSWLENGRELPGINTTISQDPESELYTISSQLDFNTTRNHTIKCLIKYGDAHVSEDFTWEKPPEDPPDSKNTLVLFGAGFGAVITVVVIVVIIKCFCKHRSCFRRNEASRETNNSLTFGPEEALAEQTVFL